(19) United States
(12) Patent Application Publication
(10) Pub. No.: US 2015/0284349 A1
(43) Pub. Date: Oct. 8, 2015

(54) EPIDITHIODIKETOPIPERAZINE COMPOUNDS, COMPOSITIONS, AND METHODS

(71) Applicant: Globavir Biosciences, Inc., Los Altos, CA (US)

(72) Inventors: Chenera Balan, Thousand Oaks, CA (US); Sumit Mahajan, Mountain View, CA (US); Usha Nagavarapu, San Jose, CA (US); Shalabh Gupta, Mountain View, CA (US)

(21) Appl. No.: 14/578,277

(22) Filed: Dec. 19, 2014

Related U.S. Application Data
(60) Provisional application No. 61/919,524, filed on Dec. 20, 2013.

(51) Int. Cl. C07D 285/15; A61K 45/06; A61K 31/548

(52) U.S. Cl. CPC C07D 285/15; A61K 31/548; A61K 45/06

(57) ABSTRACT

Epidithiodiketopiperazine compounds, pharmaceutical compositions based thereon and methods of their synthesis, as part of treating, inhibiting and reducing transcription and translation of hypoxia inducible genes are described.

### CROSS-REFERENCE

This application claims the benefit of U.S. Provisional Application Ser. No. 61/919,524, filed on Dec. 20, 2013, which application is incorporated herein by reference in its entirety.

### BACKGROUND OF THE INVENTION

Traditional cancer therapies are often limited in their efficacy because they display toxicity to normal cells and lead to drug-resistant tumors. Antiangiogenic strategies, on the other hand, have circumvented these problems by targeting the blood supply of growing tumors. While they were originally envisioned as secondary treatments to prevent the expansion of existing tumors, three decades of investigation have revealed that angiogenesis is very important for tumor growth and metastasis. The pathology of the process by which new blood vessels are formed from existing capillaries is not fully understood. However, this process is generally characterized by degradation of the basement membrane underlying an existing blood vessel. The newly created networks of highly permeable blood vessels provide efficient exit routes by which tumorigenic cells enter the bloodstream. The number of metastases formed is generally proportional to the number of invaded tumor cells. The correlation between vascular density and metastatic potential has been confirmed for many cancers with breast, ovarian, prostate, lung, and gastric carcinomas typically dominating the trend.

The decreased survival rates associated with high metastatic potential as well as the general dependence of metastasis on adequate blood supply has intensified the search for molecular targets in angiogenesis. Therapies targeting this newly formed tumor vasculature have not resulted in acquired drug resistance. Furthermore, due to the possibility of targeting replication rate of angiogenic endothelial cells, these therapies allow selective targeting of the blood supply in tumors without affecting normal blood vessels.

The high rate of cancer morbidity and mortality remains a major concern among the population in Western societies. In addition to having an impact on the cancer patients and members of their immediate families, cancer inflicts a large burden on society. The cost of cancer treatment and patient care is typically high and contributes to increased cost of health insurance and results, in turn, in a higher percentage of uninsured people and, consequently, in an increased economic burden when uninsured people become sick or injured. Cancer also causes a significant negative impact on businesses due to prolonged absences of cancer patients from work.

Although methods of cancer treatment have greatly improved over the years, many challenges, most notably relapse among cancer patients and difficulties in treating patients in advanced stages of cancer as well as with metastatic diseases or with systemic cancers such as leukemia or lymphoma, remain. For example, improved diagnostic methods combined with better surgical techniques allow oncologists to remove tumor with greater confidence, while at the same time minimizing the removal of normal tissue. As such, the recovery time for patients can be decreased and psychological impact is reduced. However, surgery is only one of the few useful tools for treating patients with localized, non-metastatic tumors or the tumors which are minimally spread.

Chemotherapy is another treatment of choice for certain types of cancers. However, chemotherapeutic methods are generally not specific for tumor cells as compared to normal cells. As a result, chemotherapy is generally associated with serious side effects and can be particularly devastating to the patient's immune system and to rapidly dividing tissues, such as tissues in liver, kidneys, gut, and epithelium.

Cancer progression is dependent on angiogenesis, or the sprouting of new blood vessels that penetrate every solid tumor. The rapid tissue proliferation which defines cancer results in a number of adaptive cellular responses, primary among which are the distinct but related processes of angiogenesis and increased glycolysis. Angiogenesis is primarily driven by several mitogenic factors such as vascular endothelial growth factor (VEGF) and its receptors play a key role. While neovascularization is essential in embryonic development, it is highly undesirable in cancers because these nascent vessels infuse tumor tissue and provide them with increased oxygenation and nutrient content for more rapid growth. Angiogenesis is particularly pernicious because it poses a double threat: not only it accelerates tumor growth, but also provides a gateway to metastasis via the newly formed vasculature. As it is metastatic growth which exerts the greatest impact on overall patient survival, angiogenesis represents a critical chemotherapeutic target. Moreover, vascular targets should not engender resistance to therapy because they are not subject to the multiple mutations which occur in malignant cells. One of the primary advantages of targeting the blood supply (vasculature) is that, unlike cells in the cancerous tissues, the cells that comprise blood vessels are genetically stable and, therefore, should have diminished resistance to therapy.

As tumor cells continue to proliferate, they are forced farther away from the blood supply carrying needed oxygen and nutrients for metabolic processes and therefore cannot attain adequate oxygen perfusion. The ensuing hypoxial results in a switch to an anaerobic metabolism which selects for cells with upregulated glycolysis Enhanced glycolytic function then leads to increased generation of lactic acid which lowers intracellular pH and can facilitate the degradation of the extracellular matrix and basement membrane, thereby promoting angiogenesis. Glycolysis confers a significant advantage in overcoming growth restraints during tumorigenesis and most primary metastatic tumors demonstrate significant upregulation of glycolytic enzymes like hexokinases 1 and 2 and glucose transporters GLUT1 and GLUT3.

Hypoxia is one of the most important hallmarks of solid tumors that plays a vital role in cell proliferation, signaling and growth. A typical neoplasm is usually devoid of blood vessels in its early stage. The rapidly proliferating cells contribute to development of hypoxia. Despite the fact that cell proliferation decreases in those parts of a tumor that are away from blood vessels, they tend to select for more aggressive cellular phenotypes. Moreover, it has been reported that the hypoxic tissue away from the blood vessels give rise to cells that have lost sensitivity to p53-mediated apoptosis.

In cells and tissues, response to hypoxia is primarily mediated by the family of hypoxia-inducible transcription factors, among which hypoxia-inducible factor 1 (HIFI.) plays a major role. It is a heterodimeric transcription factor which mediates regulation of many key genes upregulated in a hypoxic state. During normoxic conditions, the a-subunit of HIF1 is regulated by hydroxylation at proline residues 402 and 564; these modifications serve as a docking site for the von Hippel-Lindau (pVHL) protein to bind HIF1 and tag it with ubiquitin for subsequent proteasomal degradation. However, under hypoxic conditions, HIF1a accumulates, enters the nucleus and dimerizes with its beta subunit, aryl hydrocarbon receptor nuclear translocator (ARNT, or HIF11). It binds to the promoter region of hypoxia inducible genes possessing hypoxia-response elements (HREs), including VEGF, c-Met, EPO, and GLUT-1. Because low oxygen levels also preclude hydroxylation of another regulatory site at Asn803, the coactivator CREB binding protein (CBP)/p30031-33 is recruited via binding the C-terminal domain of HIF1a and promotes elevated expression levels of hypoxia-inducible genes. In many tumor cells where oncogenic mutations in RAS, SRC and HER2/NEU/ERBB2 are found, high levels of HIF1a have been detected even under well-oxygenated condition.

It has been shown that antisense construct of HIF1a eradicates in vivo a small transplanted thymic lymphoma and even increases the efficacy of immunotherapy against larger tumors. Small molecule inhibitors of microtubules, such as 2-methoxyestradiol, vincristine and paclitaxel have been shown to reduce HIFIa levels in vitro and also reduce tumor growth and vascularization. However, it is not clearly understood whether the effects shown in tumor growth reduction is due to microtubule inhibition or reduction of HIF1a levels.

HIF1a interacts primarily with the CH1 domain of CBP/300 through a series of key cysteine residues and this interaction is driven by hydrophobic forces. It was shown that the natural product chetomin (FIG. 2, vide infra), a fungal metabolite of the Chaetomium sp., demonstrated potent and specific inhibition of the HIF/p300 complex. Because p300/CBP is absolutely required for HIF1a-mediated transactivation, blocking the association of HIF1a and p300/CBP effectively downregulates transcription.

### SUMMARY OF THE INVENTION

In one aspect, the present disclosure provides a compound of Formula I

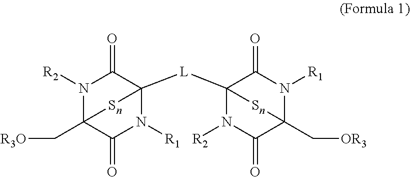

or a pharmaceutically acceptable salt thereof, wherein: each n is independently 1 or 2;
each R₁ and R₂ is independently selected from the group consisting of H, optionally substituted C₁-C₆ alkyl, optionally substituted C₃-C₇ heterocycloalkyl and optionally substituted C₃-C₇ cycloalkyl; each R₃ is independently selected from the group consisting of H, optionally substituted C₁-C₆ alkyl, PEG, —C(O)R₄, —C(O)OR₄, —C(O)NR₄, —S(O)₂R₄;
each R₄ when present is independently selected from the group consisting of optionally substituted C₁-C₆ alkyl, optionally substituted C₃-C₇ heterocycloalkyl, optionally substituted C₃-C₇ cycloalkyl, optionally substituted aryl, and optionally substituted heteroaryl; L is:

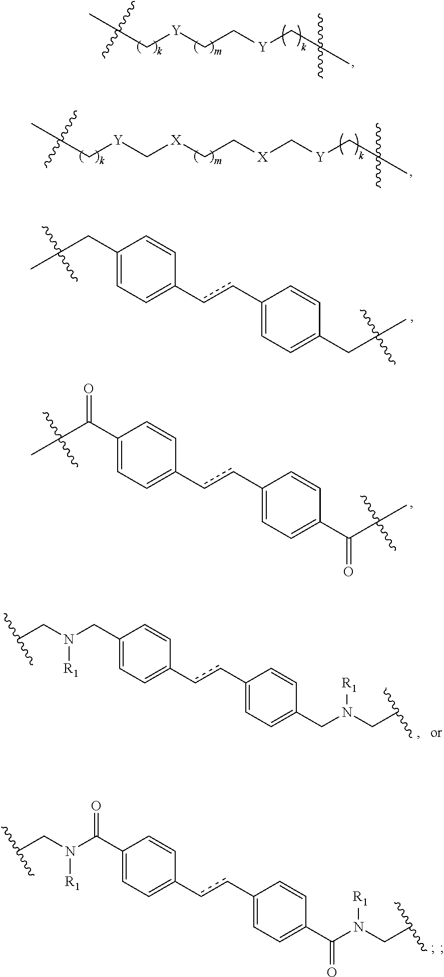

each X is independently NR₅ or O; each Y is independently selected from the group consisting of a bond, methylene, aryl and heteroaryl; [symbol] is a single or double bond; each R₅ is independently selected from the group consisting of H, optionally substituted C₁-C₆ alkyl, optionally substituted C₃-C₇ heterocycloalkyl and optionally substituted C₃-C₇ cycloalkyl; each k is independently 0, 1, 2, or 3; and m is 1, 2, 3, or 4. In some embodiments, each R₁ and each R₂ is independently selected from the group consisting of H, optionally substituted C₁-C₆ alkyl, and optionally substituted C₃-C₇ cycloalkyl, for instance an optionally substituted cyclopropyl. In some embodiments, each R₃ is independently selected from the group consisting of H, —C(O)R₄, —C(O)OR₄, —C(O)NR₄. In some embodiments, R₃ is H.

In some embodiments of the compound of Formula 1, L is

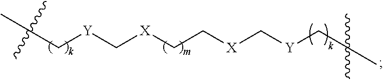

wherein each X is independently NR5 or O; each R5 is independently selected from the group consisting of H, optionally substituted C1-C6 alkyl, optionally substituted C3-C7 heterocycloalkyl and optionally substituted C3-C7 cycloalkyl; each Y is independently selected from the group consisting of a bond, methylene, aryl and heteroaryl; each k is independently 0, 1, 2, or 3; and m is 1, 2, 3, or 4. In some embodiments, L is

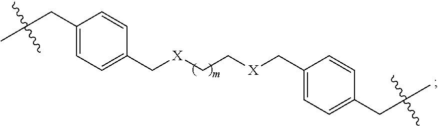

wherein each X is independently NR5 or O; and m is 1, 2, 3, or 4. In some embodiments of the compound of Formula 1, L is

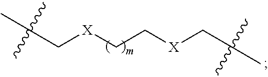

and m is 1, 2, 3, or 4. In some embodiments, each X is NR5; for instance each X is NH. In other embodiments, each X is O. In some embodiments of the compound of Formula 1, L is

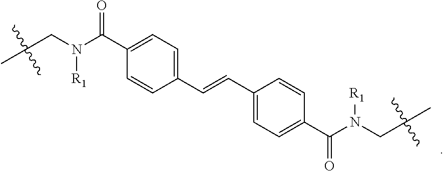

In other embodiments of the compound of Formula 1, L is

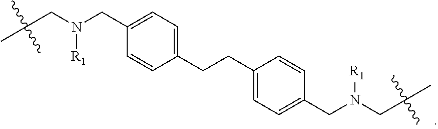

In other embodiments of the compounds of Formula 1, L is

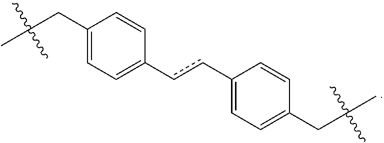

In other embodiments of the compounds of Formula 1, L is

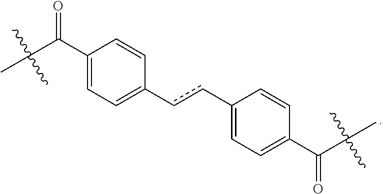

In still other embodiments of the compounds of Formula 1, L is L is

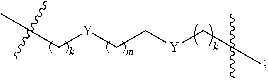

each Y is independently selected from the group consisting of a bond, methylene, aryl and heteroaryl; each k is independently 0, 1, 2, or 3; and m is 1, 2, 3, or 4. In some embodiments, Y is independently aryl or heteroaryl. For example, Y is phenyl or pyridyl. In some embodiments, each k is 1. In some embodiments, m is 2.

In some embodiments of the compound of Formula 1, R1 is H. In some embodiments of the compounds disclosed herein, the compound of Formula 1 is a compound having the structure of Formula 1a:

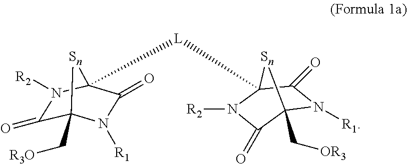

In some embodiments of the compounds disclosed herein, the compound of Formula 1 is a compound having the structure of Formula 1b:

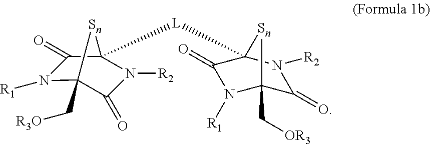

In some embodiments of the compounds disclosed herein, the compound of Formula 1 is a compound having the structure of Formula 1c:

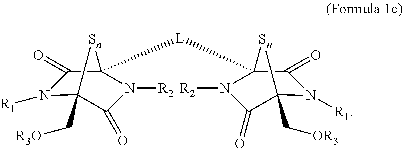

In some embodiments, the compound is selected from the group consisting of:

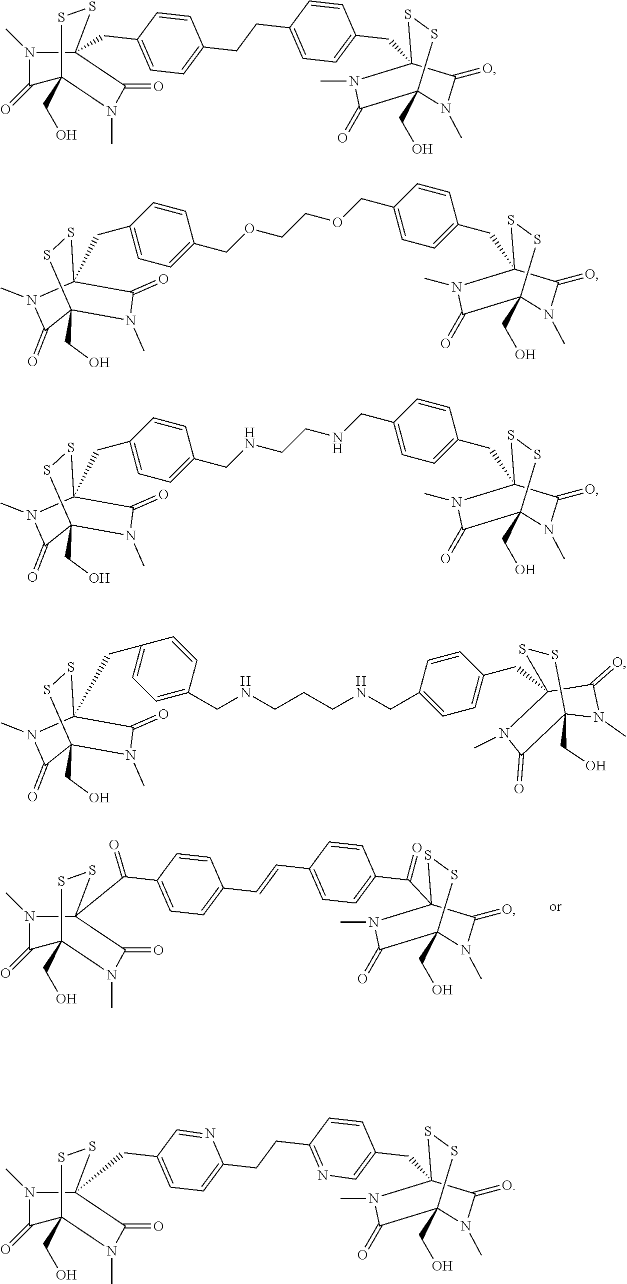

In another aspect, the present disclosure describes a pharmaceutical composition comprising at least one compound of Formula 1.

In another aspect, the present disclosure describes a method for interfering with hypoxia-induced transcriptional pathway in a cell comprising: contacting the cell with at least one compound disclosed herein. In another aspect, the present disclosure describes a method for treating breast cancer, comprising: administering to a subject in need thereof an effective amount of at least one compound disclosed herein. In another aspect, the present disclosure describes a method for treating a solid cancer, comprising: administering to a subject in need thereof an effective amount of at least one compound disclosed herein. In another aspect, the present disclosure describes a method for treating a blood cancer, comprising: administering to a subject in need thereof an effective amount of at least one compound disclosed herein. In another aspect, the present disclosure describes a method for treating a subject suffering from carcinoma in need of said treatment, comprising: administering to the subject an effective amount of at least one compound disclosed herein. In another aspect, the present disclosure describes a method for treating a subject suffering from renal cell carcinoma (RCC) in need of said treatment, comprising: administering to the subject an effective amount of at least one compound disclosed herein. In some embodiments of the methods described herein, the method further comprises administering an additional anti-cancer and/or cytotoxic agent.

In yet another aspect, the present disclosure describes a method for interfering with a protein-protein interaction between p300 and a viral protein in a cell comprising: contacting the cell with at least one compound disclosed herein. In another aspect, the present disclosure describes a method for treating a viral infection, comprising: administering to a subject in need thereof an effective amount of at least one compound disclosed herein. In some embodiments, the viral infection is an infection of human papilloma virus (HPV), hepatitis C (HCV), Hep B, or adenovirus. In some embodiments of the methods decribed herein, the method further comprises administering an additional antiviral agent.

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A shows RCC tumors (786-O cell line, derived from renal cell carcinoma of the clear cell type) in mice treated with compound 1 of the disclosure relative to mice in a control group that was not treated with the compound; FIG. 1B shows a Box-whisker diagram of the percentages of tumor volumes measured throughout a 46 day experiment with boxes representing the upper and lower quartiles and median and error bars showing maximum and minimum tumor volumes.

FIG. 2 shows intensity of the NIR signal originating from the tumor accumulated contrast agent (Xenogen IVIS 200 images) of mice treated with compound 1 of the disclosure as compared to control group

FIG. 3 shows the plasma concentration of compound 1 of the disclosure over time in an intravenous pharmacokinetic study of male BALB/c mice.

FIG. 4 shows the plasma concentration of compound 1 of the disclosure over time in an intravenous pharmacokinetic study of male and female BALB/c mice.

FIG. 5 shows the plasma concentration of compound 1 of the disclosure over time in an intravenous pharmacokinetic study of female BALB/c mice.

FIG. 6 shows the plasma concentration of compound 1 of the disclosure over time in an intravenous pharmacokinetic study of male SD rats (0.5 mg/kg b.w.).

FIG. 7 shows the plasma concentration of compound 1 of the disclosure over time in an intravenous pharmacokinetic study of male SD rats (2.5 mg/kg b.w.).

FIG. 8 shows the plasma concentration of compound 1 of the disclosure over time in an intravenous pharmacokinetic study of male SD rats (5 mg/kg b.w.).

FIG. 9 shows the plasma concentration of compound 1 of the disclosure over time in an oral pharmacokinetic study of male BALB/c mice (5 mg/kg b.w.) with SBEβCD formulation.

FIG. 10 shows the plasma concentration of compound 1 of the disclosure over time in an oral pharmacokinetic study of male and female BALB/c mice (5 mg/kg b.w.) with SBEβCD formulation.

FIG. 11 shows the plasma concentration of compound 1 of the disclosure over time in an oral pharmacokinetic study of male BALB/c mice (10 mg/kg b.w.) with SBEβCD formulation.

FIG. 12 shows the plasma concentration of compound 1 of the disclosure over time in an oral pharmacokinetic study of male and female BALB/c mice (10 mg/kg b.w.) with SBEβCD formulation.

FIG. 13 shows the plasma concentration of compound 1 of the disclosure over time in an oral pharmacokinetic study of male SD rats (10 mg/kg b.w.) with SBEβCD formulation.

### DETAILED DESCRIPTION OF THE INVENTION

As used herein, the following words and phrases are generally intended to have the meanings as set forth below, except to the extent that the context in which they are used indicates otherwise.

The following abbreviations and terms have the indicated meanings throughout:
AcOH=acetic acid
Boc=tert-butoxycarbonyl
c-=cyclo
DCC=dicyclohexylcarbodiimide
DIEA=N,N-diisopropylethylamine
DMAP=4-dimethylaminopyridine
EDC=1-ethyl-3-(3-dimethylaminopropyl)carbodiimide
eq=equivalent(s)
Et=ethyl
EtOAc or EA=ethyl acetate
EtOH=ethanol
g=gram
h or hr=hour
HBTU=O-(benzotriazol-1-yl)-N,N,N′,N′-tetramethyluronium hexafluorophosphate
HOBt=hydroxybenzotriazole
HPLC=high pressure liquid chromatography
i-=iso
kg or Kg=kilogram
L or 1=liter
LC/MS=LCMS=liquid chromatography-mass spectrometry
LRMS=low resolution mass spectrometry
m/z=mass-to-charge ratio
Me=methyl
MeOH=methanol
mg=milligram
min=minute
mL=milliliter
mmol=millimole
n-=normal
NaOAc=sodium acetate
PE=petroleum ether
Ph=phenyl
Prep=preparative
quant.=quantitative
RP-HPLC=reverse phase-high pressure liquid chromatography
rt, r.t., or RT=room temperature
s-=sec-=secondary
t-=tert-=tertiary
THF=tetrahydrofuran
TLC=thin layer chromatography
UV=ultraviolet

As used herein, when any variable occurs more than one time in a chemical formula, its definition on each occurrence is independent of its definition at every other occurrence.

As used herein, a dash (“—”) that is not between two letters or symbols is used to indicate a point of attachment for a substituent. For example, —CONH₂ is attached through the carbon atom.

As used herein, “optional” or “optionally” is meant that the subsequently described event or circumstance may or may not occur, and that the description includes instances wherein the event or circumstance occurs and instances in which it does not. For example, “optionally substituted alkyl” encompasses both “alkyl” and “substituted alkyl” as defined below. It will be understood by those skilled in the art, with respect to any group containing one or more substituents, that such groups are not intended to introduce any substitution or substitution patterns that are sterically impractical, synthetically non-feasible and/or inherently unstable.

As used herein, “alkyl” refers to straight chain and branched chain having the indicated number of carbon atoms, usually from 1 to 20 carbon atoms, for example 1 to 8 carbon atoms, such as 1 to 6 carbon atoms. For example C₁-C₆ alkyl encompasses both straight and branched chain alkyl of from 1 to 6 carbon atoms. When an alkyl residue having a specific number of carbons is named, all branched and straight chain versions having that number of carbons are intended to be encompassed; thus, for example, “butyl” is meant to include n-butyl, sec-butyl, isobutyl and t-butyl; “propyl” includes n-propyl and isopropyl. “Lower alkyl” refers to alkyl groups having one to six carbons. Examples of alkyl groups include methyl, ethyl, propyl, isopropyl, n-butyl, sec-butyl, tert-butyl, pentyl, 2-pentyl, isopentyl, neopentyl, hexyl, 2-hexyl, 3-hexyl, 3-methylpentyl, and the like. Alkylene is a subset of alkyl, referring to the same residues as alkyl, but having two points of attachment. Alkylene groups will usually have from 2 to 20 carbon atoms, for example 2 to 8 carbon atoms, such as from 2 to 6 carbon atoms. For example, C₀ alkylene indicates a covalent bond and C₁ alkylene is a methylene group.

As used herein, “alkenyl” refers to an unsaturated branched or straight-chain alkyl group having at least one carbon-carbon double bond derived by the removal of one molecule of hydrogen from adjacent carbon atoms of the parent alkyl. The group may be in either the cis or trans configuration about the double bond(s). Typical alkenyl groups include, but are not limited to, ethenyl; propenyls such as prop-1-en-1-yl, prop-1-en-2-yl, prop-2-en-1-yl (allyl), prop-2-en-2-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl; and the like. In certain embodiments, an alkenyl group has from 2 to 20 carbon atoms and in other embodiments, from 2 to 6 carbon atoms. “Lower alkenyl” refers to alkenyl groups having two to six carbons.

As used herein, “alkynyl” refers to an unsaturated branched or straight-chain alkyl group having at least one carbon-carbon triple bond derived by the removal of two molecules of hydrogen from adjacent carbon atoms of the parent alkyl. Typical alkynyl groups include, but are not limited to, ethynyl; propynyls such as prop-1-yn-1-yl, prop-2-yn-1-yl; butynyls such as but-1-yn-1-yl, but-1-yn-3-yl, but-3-yn-1-yl; and the like. In certain embodiments, an alkynyl group has from 2 to 20 carbon atoms and in other embodiments, from 3 to 6 carbon atoms. “Lower alkynyl” refers to alkynyl groups having two to six carbons.

As used herein, “cycloalkyl” refers to a non-aromatic carbocyclic ring, usually having from 3 to 7 ring carbon atoms. The ring may be saturated or have one or more carbon-carbon double bonds. Examples of cycloalkyl groups include cyclopropyl, cyclobutyl, cyclopentyl, cyclopentenyl, cyclohexyl, and cyclohexenyl, as well as bridged and caged ring groups such as norbornane.

As used herein, “alkoxy” refers to an alkyl group of the indicated number of carbon atoms attached through an oxygen bridge such as, for example, methoxy, ethoxy, propoxy, isopropoxy, n-butoxy, sec-butoxy, tert-butoxy, pentyloxy, 2-pentyloxy, isopentyloxy, neopentyloxy, hexyloxy, 2-hexyloxy, 3-hexyloxy, 3-methylpentyloxy, and the like. Alkoxy groups will usually have from 1 to 7 carbon atoms attached through the oxygen bridge. “Lower alkoxy” refers to alkoxy groups having one to six carbons.

As used herein, “acyl” refers to the groups H—C(O)—; (alkyl)-C(O)—; (cycloalkyl)-C(O)—; (aryl)-C(O)—; (heteroaryl)-C(O)—; and (heterocycloalkyl)-C(O)—, wherein the group is attached to the parent structure through the carbonyl functionality and wherein alkyl, cycloalkyl, aryl, heteroaryl, and heterocycloalkyl are as described herein. Acyl groups have the indicated number of carbon atoms, with the carbon of the keto group being included in the numbered carbon atoms. For example a C₂ acyl group is an acetyl group having the formula CH₃(C═O)—.

As used herein, “formyl” refers to the group —C(O)H.

As used herein, “alkoxycarbonyl” refers to a group of the formula (alkoxy)(C═O)— attached through the carbonyl carbon wherein the alkoxy group has the indicated number of carbon atoms. Thus a C₁-C₆ alkoxycarbonyl group is an alkoxy group having from 1 to 6 carbon atoms attached through its oxygen to a carbonyl linker

As used herein, “azido” refers to the group —N₃.

As used herein, “amino” refers to the group —NH₂.

As used herein, “mono- and di-(alkyl)amino” refers to secondary and tertiary alkyl amino groups, wherein the alkyl groups are as defined above and have the indicated number of carbon atoms. The point of attachment of the alkylamino group is on the nitrogen. Examples of mono- and di-alkylamino groups include ethylamino, dimethylamino, and methyl-propyl-amino

As used herein, “aminocarbonyl” refers to the group —CONR^{b}Re, where

R^{b} is H, optionally substituted C₁-C₆ alkyl, optionally substituted cycloalkyl, optionally substituted heterocycloalkyl, optionally substituted aryl, optionally substituted heteroaryl, or optionally substituted alkoxy; and

R^{c} is hydrogen or optionally substituted C₁-C₄ alkyl; or

R^{b} and R^{c} taken together with the nitrogen to which they are bound, form an optionally substituted 4- to 8-membered nitrogen-containing heterocycloalkyl which optionally includes 1 or 2 additional heteroatoms chosen from O, N, and S in the heterocycloalkyl ring;

where each substituted group is independently substituted with one or more substituents independently C₁-C₄ alkyl, aryl, heteroaryl, aryl-C₁-C₄ alkyl-, heteroaryl-C₁-C₄ alkyl-, C₁-C₄ haloalkyl, —OC₁-C₄ alkyl, —OC₁-C₄ alkylphenyl, —C₁-C₄ alkyl-OH, —OC₁-C₄ haloalkyl, halo, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo (as a substituent for cycloalkyl, heterocycloalkyl, or heteroaryl), —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ alkylphenyl, —C(O)C₁-C₄ haloalkyl, —OC(O)C₁-C₄ alkyl, —SO₂(C₁-C₄ alkyl), —SO₂(phenyl), —SO₂(C₁-C₄haloalkyl), —SO₂NH₂, —SO₂NH(C₁-C₄ alkyl), —SO₂NH(phenyl), —NHSO₂(C₁-C₄ alkyl), —NHSO₂(phenyl), or —NHSO₂(C₁-C₄ haloalkyl).

As used herein, “aryl” refers to: 6-membered carbocyclic aromatic rings, for example, benzene; bicyclic ring systems wherein at least one ring is carbocyclic and aromatic, for example, naphthalene, indane, and tetralin; and tricyclic ring systems wherein at least one ring is carbocyclic and aromatic, for example, fluorene.

For example, aryl includes 6-membered carbocyclic aromatic rings fused to a 4- to 8-membered heterocycloalkyl ring containing 1 or more heteroatoms chosen from N, O, and S. For such fused, bicyclic ring systems wherein only one of the rings is a carbocyclic aromatic ring, the point of attachment may be at the carbocyclic aromatic ring or the heterocycloalkyl ring. Bivalent radicals formed from substituted benzene derivatives and having the free valences at ring atoms are named as substituted phenylene radicals. Bivalent radicals derived from univalent polycyclic hydrocarbon radicals whose names end in “-yl” by removal of one hydrogen atom from the carbon atom with the free valence are named by adding “-idene” to the name of the corresponding univalent radical, e.g. a naphthyl group with two points of attachment is termed naphthylidene. Aryl, however, does not encompass or overlap in any way with heteroaryl, separately defined below. Hence, if one or more carbocyclic aromatic rings is fused with a heterocycloalkyl aromatic ring, the resulting ring system is heteroaryl, not aryl, as defined herein.

As used herein, “aryloxy” refers to the group —O-aryl.

As used herein, “aralkyl” refers to the group -alkyl-aryl.

As used herein, “carbamimidoyl” refers to the group —C(═NH)—NH2.

As used herein, “substituted carbamimidoyl” refers to the group —C(═NR^{e})—NR^{f}R^{g} where

R^{e} is hydrogen, cyano, optionally substituted alkyl, optionally substituted cycloalkyl, optionally substituted aryl, optionally substituted heteroaryl, or optionally substituted heterocycloalkyl; and

R^{f} and R^{g} are independently hydrogen optionally substituted alkyl, optionally substituted cycloalkyl, optionally substituted aryl, optionally substituted heteroaryl, or optionally substituted heterocycloalkyl,

provided that at least one of R^{e}, R^{f}, and R^{g} is not hydrogen and wherein substituted alkyl, cycloalkyl, aryl, heterocycloalkyl, and heteroaryl refer respectively to alkyl, cycloalkyl, aryl, heterocycloalkyl, and heteroaryl wherein one or more (such as up to 5, for example, up to 3) hydrogen atoms are replaced by a substituent independently —R^{a}, —OR^{b}, optionally substituted amino (including —NR^{c}COR^{b}, —NR^{c}CO₂R^{a}, —NR^{c}CONR^{b}R^{c}, —NR^{b}C(NR^{c})NR^{b}R^{c}, —NR^{b}C(NCN)NR^{b}R^{c}, and —NR^{c}SO₂R^{a}), halo, cyano, nitro, oxo (as a substituent for cycloalkyl, heterocycloalkyl, and heteroaryl), optionally substituted acyl (such as —COR^{b}), optionally substituted alkoxycarbonyl (such as —CO₂R^{b}), aminocarbonyl (such as —CONR^{b}R^{c}), —OCOR^{b}, —OCO₂R^{a}, —OCONR^{b}R^{c}, —OP(O)(OR^{b})OR^{c}, sulfanyl (such as SR^{b}), sulfinyl (such as —SOR^{a}), or sulfonyl (such as —SO₂R^{a} and —SO₂NR^{b}R^{c}),

where R^{a} is optionally substituted C1-C6 alkyl, optionally substituted aryl, or optionally substituted heteroaryl;

R^{b} is H, optionally substituted C1-C6 alkyl, optionally substituted aryl, or optionally substituted heteroaryl; and

R^{c} is hydrogen or optionally substituted C1-C4 alkyl; or

R^{b} and R^{c}, and the nitrogen to which they are attached, form an optionally substituted heterocycloalkyl group; and

where each optionally substituted group is unsubstituted or independently substituted with one or more, such as one, two, or three, substituents independently C₁-C₄ alkyl, aryl, heteroaryl, aryl-C₁-C₄ alkyl-, heteroaryl-C₁-C₄ alkyl-, C₁-C₄ haloalkyl, —OC₁-C₄ alkyl, —OC₁-C₄ alkylphenyl, —C₁-C₄ alkyl-OH, —OC₁-C₄ haloalkyl, halo, —OH, —NH₂, —C₁-C₄ alkyl-NH₂, —N(C₁-C₄ alkyl)(C₁-C₄ alkyl), —NH(C₁-C₄ alkyl), —N(C₁-C_{4 alkyl)(C}₁-C₄ alkylphenyl), —NH(C₁-C₄ alkylphenyl), cyano, nitro, oxo (as a substituent for cycloalkyl, heterocycloalkyl, or heteroaryl), —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ phenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ alkyl), —SO₂ NH(phenyl), —NHSO₂(C₁-C₄ alkyl), —NHSO₂(phenyl), or —NHSO₂(C₁-C₄ haloalkyl).

As used herein, “halo” refers to fluoro, chloro, bromo, and iodo, and the term “halogen” includes fluorine, chlorine, bromine, and iodine.

As used herein, “haloalkyl” refers to alkyl as defined above having the specified number of carbon atoms, substituted with 1 or more halogen atoms, up to the maximum allowable number of halogen atoms. Examples of haloalkyl include, but are not limited to, trifluoromethyl, difluoromethyl, 2-fluoroethyl, and penta-fluoroethyl.

As used herein, “heteroaryl” refers to: 5- to 7-membered aromatic, monocyclic rings containing one or more, for example, from 1 to 4, or in certain embodiments, from 1 to 3, heteroatoms chosen from N, O, and S, with the remaining ring atoms being carbon; bicyclic heterocycloalkyl rings containing one or more, for example, from 1 to 4, or in certain embodiments, from 1 to 3, heteroatoms chosen from N, O, and S, with the remaining ring atoms being carbon and wherein at least one heteroatom is present in an aromatic ring; and

tricyclic heterocycloalkyl rings containing one or more, for example, from 1 to 5, or in certain embodiments, from 1 to 4, heteroatoms chosen from N, O, and S, with the remaining ring atoms being carbon and wherein at least one heteroatom is present in an aromatic ring.

For example, heteroaryl includes a 5- to 7-membered heterocycloalkyl, aromatic ring fused to a 4- to 8-membered cycloalkyl or heterocycloalkyl ring. For such fused, bicyclic heteroaryl ring systems wherein only one of the rings contains one or more heteroatoms, the point of attachment may be at either ring. When the total number of S and O atoms in the heteroaryl group exceeds 1, those heteroatoms are not adjacent to one another. In certain embodiments, the total number of S and O atoms in the heteroaryl group is not more than 2. In certain embodiments, the total number of S and O atoms in the aromatic heterocycle is not more than 1. Examples of heteroaryl groups include, but are not limited to, pyridyl, pyrazinyl, pyrimidinyl, pyrazolinyl, imidazolyl, isoxazolyl, oxazolyl, thiazolyl, thiadiazolyl, tetrazolyl, thienyl, benzothiophenyl, furanyl, pyrrolyl, benzofuranyl, benzoimidazolyl, indolyl, pyridazinyl, triazolyl, quinolinyl, quinoxalinyl, pyrazolyl, and 5,6,7,8-tetrahydroisoquinolinyl. Bivalent radicals derived from univalent heteroaryl radicals whose names end in “-yl” by removal of one hydrogen atom from the atom with the free valence are named by adding “-idene” to the name of the corresponding univalent radical, e.g. a pyridyl group with two points of attachment is a pyridylidene. Heteroaryl does not encompass or overlap with aryl, cycloalkyl, or heterocycloalkyl, as defined herein.

Substituted heteroaryl also includes ring systems substituted with one or more oxide (—O⁻) substituents, such as pyridinyl N-oxides.

As used herein, “heterocycloalkyl” refers to a single, non-aromatic ring, usually with 3 to 8 ring atoms, containing at least 2 carbon atoms in addition to 1-3 heteroatoms independently chosen from oxygen, sulfur, and nitrogen, as well as combinations comprising at least one of the foregoing heteroatoms. The ring may be saturated or have one or more carbon-carbon double bonds. Suitable heterocycloalkyl groups include but are not limited to, for example, pyrrolidinyl, morpholinyl, piperidinyl, piperazinyl, azetidinyl, diazepanyl, diazocanyl, pyrrolidinyl, morpholinyl, piperidinyl, piperazinyl, imidazolidinyl, pyrazolidinyl, dihydrofuranyl, and tetrahydrofuranyl. Substituted heterocycloalkyl can also include ring systems substituted with one or more oxo (═O) or oxide (—O⁻) substituents, such as piperidinyl N-oxide, morpholinyl-N-oxide, 1-oxo-1-thiomorpholinyl and 1,1-dioxo-1-thiomorpholinyl.

“Heterocycloalkyl” also includes bicyclic ring systems wherein one non-aromatic ring, usually with 3 to 7 ring atoms, contains at least 2 carbon atoms in addition to 1-3 heteroatoms independently chosen from oxygen, sulfur, and nitrogen, as well as combinations comprising at least one of the foregoing heteroatoms; and the other ring, usually with 3 to 7 ring atoms, optionally contains 1-3 heteratoms independently chosen from oxygen, sulfur, and nitrogen and is not aromatic.

As used herein, “sulfanyl” refers to the groups: —S-(optionally substituted (C₁-C₆)alkyl), —S-(optionally substituted cycloalkyl), —S-(optionally substituted aryl), —S-(optionally substituted heteroaryl), and —S-(optionally substituted heterocycloalkyl). Hence, sulfanyl includes the group C₁-C₆ alkylsulfanyl.

As used herein, “sulfinyl” refers to the groups: —S(O)-(optionally substituted (C₁-C₆)alkyl), —S(O)-(optionally substituted cycloalkyl), —S(O)-(optionally substituted aryl), —S(O)-optionally substituted heteroaryl), —S(O)-(optionally substituted heterocycloalkyl); and —S(O)-(optionally substituted amino).

As used herein, “sulfonyl” refers to the groups: —S(O₂)-(optionally substituted (C₁-C₆)alkyl), —S(O₂)-(optionally substituted cycloalkyl), —S(O₂)-(optionally substituted aryl), —S(O₂)-(optionally substituted heteroaryl), —S(O₂)-(optionally substituted heterocycloalkyl), and —S(O₂)-(optionally substituted amino)

As used herein, “substituted” refers to any one or more hydrogens on the designated atom or group is replaced with a selection from the indicated group, provided that the designated atom's normal valence is not exceeded. When a substituent is oxo (i.e. ═O) then 2 hydrogens on the atom are replaced. Combinations of substituents and/or variables are permissible only if such combinations result in stable compounds or useful synthetic intermediates. A stable compound or stable structure is meant to imply a compound that is sufficiently robust to survive isolation from a reaction mixture, and subsequent formulation as an agent having at least practical utility. Unless otherwise specified, substituents are named into the core structure. For example, it is to be understood that when (cycloalkyl)alkyl is listed as a possible substituent, the point of attachment of this substituent to the core structure is in the alkyl portion.

As used herein, the terms “substituted” alkyl, cycloalkyl, aryl, heterocycloalkyl, and heteroaryl, unless otherwise expressly defined, refer respectively to alkyl, cycloalkyl, aryl, heterocycloalkyl, and heteroaryl wherein one or more (such as up to 5, for example, up to 3) hydrogen atoms are replaced by a substituent independently —R^{a}, —OR^{b}, optionally substituted amino (including —NR^{c}COR^{b}, —NR^{c}CO₂R^{a}, —NR^{c}CONR^{b}R^{c}, —NR^{b}C(NR^{c})NR^{b}R^{c}, —NR^{b}C(NCN)NR^{b}R^{c}, and —NR^{c}SO₂R^{a}), halo, cyano, azido, nitro, oxo (as a substituent for cycloalkyl or heterocycloalkyl), optionally substituted acyl (such as —COR^{b}), optionally substituted alkoxycarbonyl (such as —CO₂R^{b}), aminocarbonyl (such as —CONR^{b}R^{c}), —OCOR^{b}, —OCO₂R^{a}, —OCONR^{b}R^{c}, —OP(O)(O^{Rb})OR^{c}′ sulfanyl (such as SR^{b}), sulfinyl (such as —SOR^{a})′ or sulfonyl (such as —SO₂R^{a} and —SO₂NR^{b}R^{c}),

where R^{a} is optionally substituted C₁-C₆ alkyl, optionally substituted cycloalkyl, optionally substituted heterocycloalkyl, optionally substituted alkenyl, optionally substituted alkynyl, optionally substituted aryl, or optionally substituted heteroaryl; R^{b} is hydrogen, optionally substituted C₁-C₆ alkyl, optionally substituted cycloalkyl, optionally substituted heterocycloalkyl, optionally substituted aryl, or optionally substituted heteroaryl; and

R^{c} is hydrogen or optionally substituted C₁-C₄ alkyl; or

R^{b} and R^{c}, and the nitrogen to which they are attached, form an optionally substituted heterocycloalkyl group; and

where each optionally substituted group is unsubstituted or independently substituted with one or more, such as one, two, or three, substituents independently C₁-C₄ alkyl, aryl, heteroaryl, aryl-C₁-C₄ alkyl-, heteroaryl-C₁-C₄ alkyl-, C₁-C₄ haloalkyl, —OC₁-C₄ alkyl, —OC₁-C₄ alkylphenyl, —C₁-C₄ alkyl-OH, —OC₁-C₄ haloalkyl, halo, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo (as a substituent for cycloalkyl or heterocycloalkyl), —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ alkylphenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), or —NHSO₂(C₁-C₄ haloalkyl).

As used herein, “substituted acyl” refers to the groups (substituted alkyl)-C(O)—; (substituted cycloalkyl)-C(O)—; (substituted aryl)-C(O)—; (substituted heteroaryl)-C(O)—; and (substituted heterocycloalkyl)-C(O)—, wherein the group is attached to the parent structure through the carbonyl functionality and wherein substituted alkyl, cycloalkyl, aryl, heteroaryl, and heterocycloalkyl, refer respectively to alkyl, cycloalkyl, aryl, heteroaryl, and heterocycloalkyl wherein one or more (such as up to 5, for example, up to 3) hydrogen atoms are replaced by a substituent independently —R^{a}, —OR^{b}, optionally substituted amino (including —NR^{c}COR^{b}, —NR^{c}CO₂R^{a}, —NR^{c}CONR^{b}R^{c}, —NR^{b}C(NR^{c})NR^{b}R^{c}, —NR^{b}C(NCN)NR^{b}R^{c}, and —NR^{c}SO₂R^{a}), halo, cyano, nitro, oxo (as a substituent for cycloalkyl or heterocycloalkyl), optionally substituted acyl (such as —COR^{b}), optionally substituted alkoxycarbonyl (such as —CO₂R^{b}), aminocarbonyl (such as —CONR^{b}R^{c}), —OCOR^{b}, —OCO₂R^{a}, —OCONR^{b}R^{c}, —OP(O)(OR^{b})OR^{c}, sulfanyl (such as SR^{b}), sulfinyl (such as —SOR^{a}), or sulfonyl (such as —SO₂R^{a} and —SO₂NR^{b}R^{e}),

where R^{a} is optionally substituted C₁-C₆ alkyl, optionally substituted alkenyl, optionally substituted alkynyl, optionally substituted aryl, or optionally substituted heteroaryl;

R^{b} is H, optionally substituted C₁-C₆ alkyl, optionally substituted cycloalkyl, optionally substituted heterocycloalkyl, optionally substituted aryl, or optionally substituted heteroaryl; and

R^{c} is hydrogen or optionally substituted C₁-C₄ alkyl; or

R^{b} and R^{c}, and the nitrogen to which they are attached, form an optionally substituted heterocycloalkyl group; and

where each optionally substituted group is unsubstituted or independently substituted with one or more, such as one, two, or three, substituents independently C₁-C₄ alkyl, aryl, heteroaryl, aryl-C₁-C₄ alkyl-, heteroaryl-C₁-C₄ alkyl-, C₁-C₄ haloalkyl, —OC₁-C₄ alkyl, —OC₁-C₄ alkylphenyl, —C₁-C₄ alkyl-OH, —OC₁-C₄ haloalkyl, halo, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo (as a substituent for cycloalkyl or heterocycloalkyl), —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ alkylphenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), or —NHSO₂(C₁-C₄ haloalkyl).

As used herein, “substituted alkoxy” refers to alkoxy wherein the alkyl constituent is substituted (i.e. —O-(substituted alkyl)) wherein “substituted alkyl” refers to alkyl wherein one or more (such as up to 5, for example, up to 3) hydrogen atoms are replaced by a substituent independently —R^{a}, —OR^{b}, optionally substituted amino (including —NR^{c}COR^{b}, —NR^{c}CO₂R^{a}, —NR^{c}CONR^{b}R^{c}, —NR^{b}C(NR^{c})NR^{b}R^{c}, —NR^{b}C(NCN)NR^{b}R^{c}, and —NR^{c}SO₂R^{a}), halo, cyano, nitro, oxo (as a substituent for cycloalkyl or heterocycloalkyl), optionally substituted acyl (such as —COR^{b}), optionally substituted alkoxycarbonyl (such as —CO₂R^{b}), aminocarbonyl (such as —CONR^{b}R^{c}), —OCOR^{b}, —OCO₂R^{a}, —OCONR^{b}R^{c}, —OP(O)(OR^{b})OR^{c}, sulfanyl (such as SR^{b}), sulfinyl (such as —SOR^{a}), and sulfonyl (such as —SO₂R^{a} and —SO₂NR^{b}R^{c}),

where R^{a} is optionally substituted C₁-C₆ alkyl, optionally substituted alkenyl, optionally substituted alkynyl, optionally substituted aryl, or optionally substituted heteroaryl;

R^{b} is H, optionally substituted C₁-C₆ alkyl, optionally substituted cycloalkyl, optionally substituted heterocycloalkyl, optionally substituted aryl, or optionally substituted heteroaryl; and

R^{c} is hydrogen or optionally substituted C₁-C₄ alkyl; or

R^{b} and R^{c}, and the nitrogen to which they are attached, form an optionally substituted heterocycloalkyl group; and

where each optionally substituted group is unsubstituted or independently substituted with one or more, such as one, two, or three, substituents independently C₁-C₄ alkyl, aryl, heteroaryl, aryl-C₁-C₄ alkyl-, heteroaryl-C₁-C₄ alkyl-, C₁-C₄ haloalkyl, —OC₁-C₄ alkyl, —OC₁-C₄ alkylphenyl, —C₁-C₄ alkyl-OH, —OC₁-C₄ haloalkyl, halo, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo (as a substituent for cycloalkyl or heterocycloalkyl), —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ alkylphenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), or —NHSO₂ (C₁-C₄ haloalkyl).

In some embodiments, a substituted alkoxy group is “polyalkoxy” or —O-(optionally substituted alkylene)-(optionally substituted alkoxy), and includes groups such as —OCH₂CH₂OCH₃, and residues of glycol ethers such as polyethyleneglycol, and —O(CH₂CH₂O)ₓCH₃, where x is an integer of 2-20, such as 2-10, and for example, 2-5. Another substituted alkoxy group is hydroxyalkoxy or —OCH₂(CH₂)_{y}OH, where y is an integer of 1-10, such as 1-4.

As used herein, “substituted alkoxycarbonyl” refers to the group (substituted alkyl)-O—C(O)— wherein the group is attached to the parent structure through the carbonyl functionality and wherein substituted refers to alkyl wherein one or more (such as up to 5, for example, up to 3) hydrogen atoms are replaced by a substituent independently —R^{a}, —OR^{b}, optionally substituted amino (including —NR^{c}COR^{b}, —NR^{c}CO₂R^{a}, —NR^{c}CONR^{b}R^{c}, —NR^{b}C(NR^{c})NR^{b}R^{c}, —NR^{b}C(NCN)NR^{b}R^{c}, and —NR^{c}SO₂R^{a}), halo, cyano, nitro, oxo (as a substituent for cycloalkyl or heterocycloalkyl), optionally substituted acyl (such as —COR^{b}), optionally substituted alkoxycarbonyl (such as —CO₂R^{b}), aminocarbonyl (such as —CONR^{b}R^{c}), —OCOR^{b}, —OCO₂R^{a}, —OCONR^{b}R^{c}, —OP(O)(OR^{b})OR^{c}, sulfanyl (such as SR^{b}), sulfinyl (such as —SOR^{a}), and sulfonyl (such as —SO₂R^{a} and —SO₂NR^{b}R^{e}),

where R^{a} is optionally substituted C₁-C₆ alkyl, optionally substituted alkenyl, optionally substituted alkynyl, optionally substituted aryl, or optionally substituted heteroaryl;

R^{b} is H, optionally substituted C₁-C₆ alkyl, optionally substituted cycloalkyl, optionally substituted heterocycloalkyl, optionally substituted aryl, or optionally substituted heteroaryl; and

R^{c} is hydrogen or optionally substituted C₁-C₄ alkyl; or

R^{b} and R^{c}, and the nitrogen to which they are attached, form an optionally substituted heterocycloalkyl group; and

where each optionally substituted group is unsubstituted or independently substituted with one or more, such as one, two, or three, substituents independently C₁-C₄ alkyl, aryl, heteroaryl, aryl-C₁-C₄ alkyl-, heteroaryl-C₁-C₄ alkyl-, C₁-C₄ haloalkyl, —OC₁-C₄ alkyl, —OC₁-C₄ alkylphenyl, —C₁-C₄ alkyl-OH, —OC₁-C₄ haloalkyl, halo, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo (as a substituent for cycloalkyl or heterocycloalkyl), —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ alkylphenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), or —NHSO₂(C₁-C₄ haloalkyl).

As used herein, “substituted amino” refers to the group —NHR^{d} or —NR^{d}Re wherein R^{d} is hydroxyl, formyl, optionally substituted alkoxy, optionally substituted alkyl, optionally substituted cycloalkyl, optionally substituted acyl, optionally substituted carbamimidoyl, aminocarbonyl, optionally substituted aryl, optionally substituted heteroaryl, optionally substituted heterocycloalkyl, optionally substituted alkoxycarbonyl, sulfinyl and sulfonyl, and wherein R^{e} is chosen from optionally substituted alkyl, optionally substituted cycloalkyl, optionally substituted aryl, optionally substituted heteroaryl, or optionally substituted heterocycloalkyl, and wherein substituted alkyl, cycloalkyl, aryl, heterocycloalkyl, and heteroaryl refer respectively to alkyl, cycloalkyl, aryl, heterocycloalkyl, and heteroaryl wherein one or more (such as up to 5, for example, up to 3) hydrogen atoms are replaced by a substituent independently —R^{a}, —OR^{b}, optionally substituted amino (including —NR^{c}COR^{b}, —NR^{c}CO₂R^{a}, —NR^{c}CONR^{b}R^{c}, —NR^{b}C(NR^{c})NR^{b}R^{c}, —NR^{b}C(NCN)NR^{b}R^{c}, and —NR^{c}SO₂R^{a}), halo, cyano, nitro, oxo (as a substituent for cycloalkyl or heterocycloalkyl), optionally substituted acyl (such as —COR^{b}), optionally substituted alkoxycarbonyl (such as —CO₂R^{b}), aminocarbonyl (such as —CONR^{b}R^{c}), —OCOR^{b}, —OCO₂R^{a}, —OCONR^{b}R^{c}, —OP(O)(OR^{b})OR^{c}, sulfanyl (such as SR^{b}), sulfinyl (such as —SOR^{a}), or sulfonyl (such as —SO₂R^{a} and —SO₂NR^{b}R^{c}),

wherein R^{a} is optionally substituted C₁-C₆ alkyl, optionally substituted alkenyl, optionally substituted alkynyl, optionally substituted aryl, or optionally substituted heteroaryl;

R^{b} is H, optionally substituted C₁-C₆ alkyl, optionally substituted cycloalkyl, optionally substituted heterocycloalkyl, optionally substituted aryl, or optionally substituted heteroaryl; and

R^{c} is hydrogen or optionally substituted C₁-C₄ alkyl; or R^{b} and R^{c}, and the nitrogen to which they are attached, form an optionally substituted heterocycloalkyl group; and wherein each optionally substituted group is unsubstituted or independently substituted with one or more, such as one, two, or three, substituents independently chosen from C₁-C₄ alkyl, aryl, heteroaryl, aryl-C₁-C₄ alkyl-, heteroaryl-C₁-C₄ alkyl-, C₁-C₄ haloalkyl, —OC₁-C₄ alkyl, —OC₁-C₄ alkylphenyl, —C₁-C₄ alkyl-OH, —OC₁-C₄ haloalkyl, halo, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo (as a substituent for cycloalkyl or heterocycloalkyl), —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ alkylphenyl, —C(O)C₁-C₄ haloalkyl, —OC(O)C₁-C₄ alkyl, —SO₂(C₁-C₄ alkyl), —SO₂(phenyl), —SO₂(C₁-C₄haloalkyl), —SO₂NH₂, —SO₂NH(C₁-C₄ alkyl), —SO₂NH(phenyl), —NHSO₂(C₁-C₄ alkyl), —NHSO₂(phenyl), or —NHSO₂(C₁-C₄ haloalkyl); and

wherein optionally substituted acyl, optionally substituted alkoxycarbonyl, sulfinyl and sulfonyl are as defined herein.

The term “substituted amino” also refers to N-oxides of the groups —NHR^{d}, and NR^{d}R^{d} each as described above. N-oxides can be prepared by treatment of the corresponding amino group with, for example, hydrogen peroxide or m-chloroperoxybenzoic acid. The person skilled in the art is familiar with reaction conditions for carrying out the N-oxidation.

Compounds described herein include, but are not limited to, their optical isomers, racemates, and other mixtures thereof. In those situations, the single enantiomers or diastereomers, i.e., optically active forms, can be obtained by asymmetric synthesis or by resolution of the racemates. Resolution of the racemates can be accomplished, for example, by conventional methods such as crystallization in the presence of a resolving agent, or chromatography, using, for example a chiral high-pressure liquid chromatography (HPLC) column. In addition, compounds include Z- and E-forms (or cis- and trans-forms) of compounds with carbon-carbon double bonds. Where compounds described herein exist in various tautomeric forms, the term “compound” is intended to include all tautomeric forms of the compound.

Compounds of Formula I also include crystalline and amorphous forms of those compounds, including, for example, polymorphs, pseudopolymorphs, solvates (including hydrates), unsolvated polymorphs (including anhydrates), conformational polymorphs, and amorphous forms of the compounds, as well as mixtures thereof. “Crystalline form,” “polymorph,” and “novel form” may be used interchangeably herein, and are meant to include all crystalline and amorphous forms of the compound, including, for example, polymorphs, pseudopolymorphs, solvates (including hydrates), unsolvated polymorphs (including anhydrates), conformational polymorphs, and amorphous forms, as well as mixtures thereof, unless a particular crystalline or amorphous form is referred to. Similarly, “pharmaceutically acceptable forms” of compounds of Formula I also include crystalline and amorphous forms of those compounds, including, for example, polymorphs, pseudopolymorphs, solvates (including hydrates), unsolvated polymorphs (including anhydrates), conformational polymorphs, and amorphous forms of the pharmaceutically acceptable salts, as well as mixtures thereof.

A “solvate” is formed by the interaction of a solvent and a compound. The term “compound” is intended to include solvates of compounds. Similarly, “pharmaceutically acceptable salts” includes solvates of pharmaceutically acceptable salts. Suitable solvates are pharmaceutically acceptable solvates, such as hydrates, including monohydrates and hemi-hydrates.

Compounds of Formula I also include other pharmaceutically acceptable forms of the recited compounds, including chelates, non-covalent complexes, prodrugs, and mixtures thereof.

A “chelate” is formed by the coordination of a compound to a metal ion at two (or more) points. The term “compound” is intended to include chelates of compounds. Similarly, “pharmaceutically acceptable salts” includes chelates of pharmaceutically acceptable salts.

A “non-covalent complex” is formed by the interaction of a compound and another molecule wherein a covalent bond is not formed between the compound and the molecule. For example, complexation can occur through van der Waals interactions, hydrogen bonding, and electrostatic interactions (also called ionic bonding). Such non-covalent complexes are included in the term “compound”. Similarly, pharmaceutically acceptable salts include “non-covalent complexes” of pharmaceutically acceptable salts.

The term “hydrogen bond” refers to a form of association between an electronegative atom (also known as a hydrogen bond acceptor) and a hydrogen atom attached to a second, relatively electronegative atom (also known as a hydrogen bond donor). Suitable hydrogen bond donor and acceptors are well understood in medicinal chemistry.

“Hydrogen bond acceptor” refers to a group comprising an oxygen or nitrogen, such as an oxygen or nitrogen that is sp²-hybridized, an ether oxygen, or the oxygen of a sulfoxide or N-oxide.

The term “hydrogen bond donor” refers to an oxygen, nitrogen, or heteroaromatic carbon that bears a hydrogen group containing a ring nitrogen or a heteroaryl group containing a ring nitrogen.

The compounds disclosed herein can be used in different enriched isotopic forms, e.g., enriched in the content of ²H, ³H, ¹¹C, ¹³C and/or ¹⁴C. In one particular embodiment, the compound is deuterated at least one position. Such deuterated forms can be made by the procedure described in U.S. Pat. Nos. 5,846,514 and 6,334,997. As described in U.S. Pat. Nos. 5,846,514 and 6,334,997, deuteration can improve the efficacy and increase the duration of action of drugs.

Deuterium substituted compounds can be synthesized using various methods such as described in: Dean, Dennis C.; Editor. Recent Advances in the Synthesis and Applications of Radiolabeled Compounds for Drug Discovery and Development. [In: Cliff., Pharm. Des., 2000; 6(10)] 2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

“Pharmaceutically acceptable salts” include, but are not limited to salts with inorganic acids, such as hydrochlorate, phosphate, diphosphate, hydrobromate, sulfate, sulfinate, nitrate, and like salts; as well as salts with an organic acid, such as malate, maleate, fumarate, tartrate, succinate, citrate, acetate, lactate, methanesulfonate, p-toluenesulfonate, 2-hydroxyethylsulfonate, benzoate, salicylate, stearate, and alkanoate such as acetate, HOOC—(CH₂)ₙ—COOH where n is 0-4, and like salts. Similarly, pharmaceutically acceptable cations include, but are not limited to sodium, potassium, calcium, aluminum, lithium, and ammonium.

In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare non-toxic pharmaceutically acceptable addition salts.

“Prodrugs” described herein include any compound that becomes a compound of Formula I when administered to a subject, e.g., upon metabolic processing of the prodrug. Similarly, “pharmaceutically acceptable salts” includes “prodrugs” of pharmaceutically acceptable salts. Examples of prodrugs include derivatives of functional groups, such as a carboxylic acid group, in the compounds of Formula I. Exemplary prodrugs of a carboxylic acid group include, but are not limited to, carboxylic acid esters such as alkyl esters, hydroxyalkyl esters, arylalkyl esters, and aryloxyalkyl esters. Other exemplary prodrugs include lower alkyl esters such as ethyl ester, acyloxyalkyl esters such as pivaloyloxymethyl (POM), glycosides, and ascorbic acid derivatives.

Other exemplary prodrugs include amides of carboxylic acids. Exemplary amide prodrugs include metabolically labile amides that are formed, for example, with an amine and a carboxylic acid. Exemplary amines include NH₂, primary, and secondary amines such as NHRX, and NRXRY, wherein Rx is hydrogen, (C₁-C₁₈)-alkyl, (C₃-C₇)-cycloalkyl, (C₃-C₇)-cycloalkyl-(C₁-C₄)-alkyl-, (C₆-C₁₄)-aryl which is unsubstituted or substituted by a residue (C₁-C₂)-alkyl, (C₁-C₂)-alkoxy, fluoro, or chloro; heteroaryl-, (C₆-C₁₄)-aryl-(C₁-C₄)-alkyl where aryl is unsubstituted or substituted by a residue (C₁-C₂)-alkyl, (C₁-C₂)-alkoxy, fluoro, or chloro; or heteroaryl-(C₁-C₄)-alkyl- and in which R^{y} has the meanings indicated for R^{x} with the exception of hydrogen or wherein R^{x} and R^{y}, together with the nitrogen to which they are bound, form an optionally substituted 4- to 7-membered heterocycloalkyl ring which optionally includes one or two additional heteroatoms chosen from nitrogen, oxygen, and sulfur. A discussion of prodrugs is provided in T. Higuchi and V. Stella, Pro-drugs as Novel Delivery Systems, Vol. 14 of the A.C.S. Symposium Series, in Edward B. Roche, ed., Bioreversible Carriers in Drug Design, American Pharmaceutical Association and Pergamon Press, 1987, and in Design of Prodrugs, ed. H. Bundgaard, Elsevier, 1985.

As used herein, the terms “group”, “radical” or “fragment” are synonymous and are intended to indicate functional groups or fragments of molecules attachable to a bond or other fragments of molecules.

As used herein, the term “leaving group” refers to the meaning conventionally associated with it in synthetic organic chemistry, i.e., an atom or group displaceable under nucleophilic displacement conditions. Examples of leaving groups include, but are not limited to, dimethylhydroxylamino (e.g. Weinreb amide), halogen, alkane- or arylsulfonyloxy, such as methanesulfonyloxy, ethanesulfonyloxy, thiomethyl, benzenesulfonyloxy, tosyloxy, and thienyloxy, dihalophosphinoyloxy, optionally substituted benzyloxy, isopropyloxy, acyloxy, and the like.

As used herein, the term “protective group” or “protecting group” refers to a group which selectively blocks one reactive site in a multifunctional compound such that a chemical reaction can be carried out selectively at another unprotected reactive site in the meaning conventionally associated with it in synthetic chemistry. Certain processes of this invention rely upon the protective groups to block certain reactive sites present in the reactants. Examples of protecting groups can be found in Wuts et al., Green's Protective Groups in Organic Synthesis, (J. Wiley, 4th ed. 2006).

As used herein, the term “deprotection” or “deprotecting” refers to a process by which a protective group is removed after a selective reaction is completed. Certain protective groups may be preferred over others due to their convenience or relative ease of removal. Without being limiting, deprotecting reagents for protected amino or anilino group include strong acid such as trifluoroacetic acid (TFA), concentrated HCl, H₂SO₄, or HBr, and the like.

As used herein, “modulation” refers to a change in activity as a direct or indirect response to the presence of a chemical entity as described herein, relative to the activity of in the absence of the chemical entity. The change may be an increase in activity or a decrease in activity, and may be due to the direct interaction of the compound with the a target or due to the interaction of the compound with one or more other factors that in turn affect the target's activity. For example, the presence of the chemical entity may, for example, increase or decrease the target activity by directly binding to the target, by causing (directly or indirectly) another factor to increase or decrease the target activity, or by (directly or indirectly) increasing or decreasing the amount of target present in the cell or organism.

As used herein, “active agent” is used to indicate a chemical entity which has biological activity. In certain embodiments, an “active agent” is a compound having pharmaceutical utility. For example an active agent may be an anti-cancer therapeutic.

As used herein, “significant” refers to any detectable change that is statistically significant in a standard parametric test of statistical significance such as Student's T-test, where p<0.05.

As used herein, a “pharmaceutically acceptable” component is one that is suitable for use with humans and/or animals without undue adverse side effects (such as toxicity, irritation, and allergic response) commensurate with a reasonable benefit/risk ratio.

As used herein, “therapeutically effective amount” of a chemical entity described herein refers to an amount effective, when administered to a human or non-human subject, to provide a therapeutic benefit such as amelioration of symptoms, slowing of disease progression, or prevention of disease.

“Treating” or “treatment” encompasses administration of at least one compound of Formula I, or a pharmaceutically acceptable salt thereof, to a mammalian subject, particularly a human subject, in need of such an administration and includes (i) arresting the development of clinical symptoms of the disease, such as cancer, (ii) bringing about a regression in the clinical symptoms of the disease, such as cancer, and/or (iii) prophylactic treatment for preventing the onset of the disease, such as cancer.

As used herein, the terms “cancer”, “hyperproliferative” and “neoplastic” refer to cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness. A metastatic tumor can arise from a multitude of primary tumor types, including but not limited to those of breast, lung, liver, colon and ovarian origin. “Pathologic hyperproliferative” cells occur in disease states characterized by malignant tumor growth. Examples of non-pathologic hyperproliferative cells include proliferation of cells associated with wound repair. Examples of cellular proliferative and/or differentiative disorders include cancer, e.g., carcinoma, sarcoma, or metastatic disorders. In some embodiments, compounds are novel therapeutic agents for controlling breast cancer, ovarian cancer, colon cancer, lung cancer, metastasis of such cancers and the like.

As used herein, “subject” refers to a mammal that has been or will be the object of treatment, observation or experiment. The methods described herein can be useful in both human therapy and veterinary applications. In some embodiments, the subject is a human.

The term “mammal” is intended to have its standard meaning, and encompasses humans, dogs, cats, sheep, and cows, for example.

### A. Compounds

In one aspect, provided is a compound of Formula 1

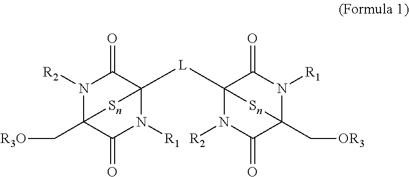

or a pharmaceutically acceptable salt thereof, wherein

each n is independently 1 or 2;
each R₁ and R₂ is independently selected from the group consisting of H, optionally substituted C₁-C₆ alkyl, optionally substituted C₃-C₇ heterocycloalkyl and optionally substituted C₃-C₇ cycloalkyl;
each R₃ is independently selected from the group consisting of H, optionally substituted C₁-C₆ alkyl, PEG, —C(O)R₄, —C(O)OR₄, —C(O)NR₄, —S(O)₂R₄;
each R₄ when present is independently selected from the group consisting of optionally substituted C₁-C₆ alkyl, optionally substituted C₃-C₇ heterocycloalkyl, optionally substituted C₃-C₇ cycloalkyl, optionally substituted aryl, and optionally substituted heteroaryl;

### L is:

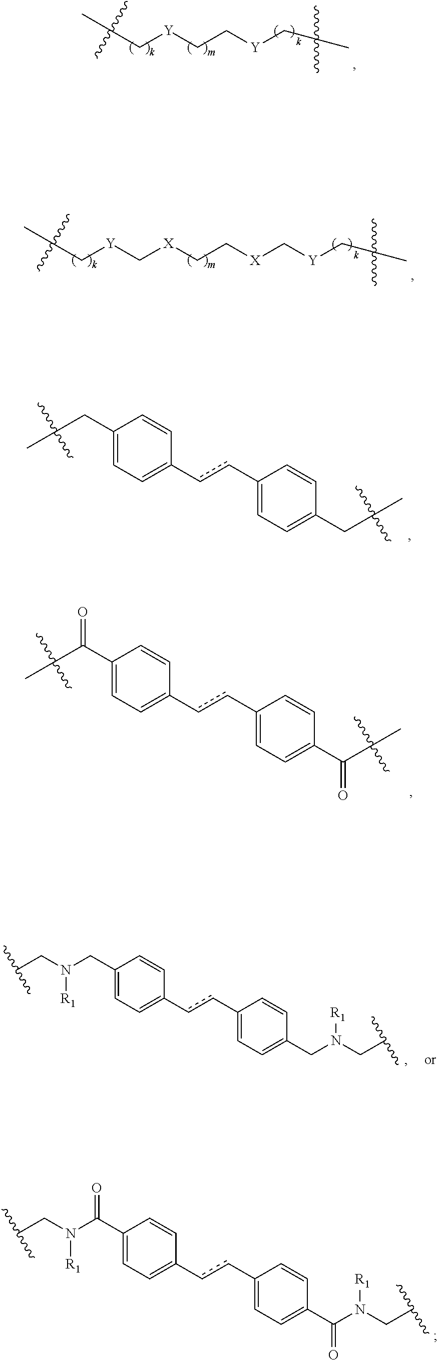

each X is independently NR₅ or O;
each Y is independently selected from the group consisting of a bond, methylene, aryl and heteroaryl; [symbol]
is a single or double bond;
each R₅ is independently selected from the group consisting of H, optionally substituted C₁-C₆ alkyl, optionally substituted C₃-C₇ heterocycloalkyl and optionally substituted C₃-C₇ cycloalkyl;
each k is independently 0, 1, 2, or 3;
and m is 1, 2, 3, or 4.

In some embodiments, each R₁ and each R₂ is independently selected from the group consisting of H, optionally substituted C₁-C₆ alkyl, and optionally substituted C₃-C₇ cycloalkyl. In some embodiments, each R₁ and each R₂ is independently selected from the group consisting of H, optionally substituted C₁-C₆ alkyl, and optionally substituted cyclopropyl. In some embodiments, each R₁ and each R₂ is independently H or optionally substituted C₁-C₆ alkyl. In some embodiments, each R₁ and each R₂ is independently H or C₁-C₆ alkyl. In some embodiments, each R₁ and each R₂ is optionally substituted C₁-C₆ alkyl. In some embodiments, each R₁ and each R₂ is selected from the group consisting of methyl, ethyl, and cyclopropyl.

In some embodiments, each R₃ is independently selected from the group consisting of H, optionally substituted C₁-C₆ alkyl, PEG, —C(O)R₄, —C(O)OR₄, —C(O)NR₄, —S(O)₂R₄; each R₄ when present is independently selected from the group consisting of optionally substituted C₁-C₆ alkyl, optionally substituted C₃-C₇ heterocycloalkyl, optionally substituted C₃-C₇ cycloalkyl, optionally substituted aryl, and optionally substituted heteroaryl. In some embodiments, each R₃ is independently selected from the group consisting of H, —C(O)R₄, —C(O)OR₄, —C(O)NR₄. In some embodiments, each R₃ is H. In some embodiments, each R₃ is selected from H and a metabolically labile group designed to increase the absorption, the distribution, or a combination thereof of the active compound in vivo. In some embodiments, at least one R₃ is selected as a prodrug. In some embodiments, at least one R₃ is an acetate group.

In some embodiments, n is 2; each R₃ is independently H or —C(O)R₄; each R₁ and R₂ is independently selected from the group consisting of H, optionally substituted C₁-C₆ alkyl, and optionally substituted C₃-C₇ cycloalkyl.

In some embodiments, n is 2; each R₃ is independently H or —C(O)R₄; each R₁ and R₂ is independently selected from the group consisting of H, C₁-C₆ alkyl, and C₃-C₇ cycloalkyl. In some embodiments, n is 2; each R₃ is independently H or —C(O)R₄; each R₁ and R₂ is independently selected from the group consisting of H, C₁-C₆ alkyl, and C₃-C₄ cycloalkyl. In some embodiments, n is 2; each R₃ is H; each R₁ and R₂ is independently selected from the group consisting of H, C₁-C₆ alkyl, and C₃-C₄ cycloalkyl. In some embodiments, n is 2; each R₃ is H; each R₁ and R₂ is independently selected from the group consisting of H, methyl, ethyl, propyl, and cyclopropyl.

In some embodiments, L is

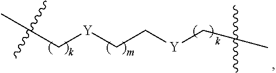

each Y is independently selected from the group consisting of a bond, methylene, aryl and heteroaryl; each k is independently 0, 1, 2, or 3; and m is 1, 2, 3, or 4.

In some embodiments, L is

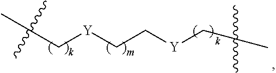

each Y is independently selected from the group consisting of a bond, methylene, aryl and heteroaryl; each k is independently 0, 1, 2, or 3; and m is 1, 2, 3, or 4; n is 2; each R₃ is H; each R₁ and R₂ is independently selected from the group consisting of H, C₁-C₆ alkyl, and C₃-C₄ cycloalkyl.

In some embodiments, L is

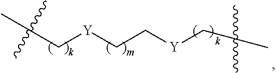

each Y is independently aryl or heteroaryl; each k is independently 0, 1, 2, or 3; and m is 1, 2, 3, or 4.

In some embodiments, L is

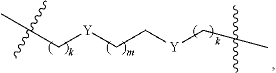

each Y is independently aryl or heteroaryl; each k is 1; and m is 1, 2, 3, or 4.

In some embodiments, L is

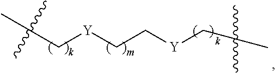

each Y is independently aryl or heteroaryl; each k is 1; and m 2. In some embodiments, Y is phenyl. In some embodiments, Y is pyridyl; n is 2; each R₃ is H; each R₁ and R₂ is independently selected from the group consisting of H, C₁-C₆ alkyl, and C₃-C₄ cycloalkyl.

In some embodiments, L is

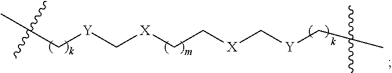

each X is independently NR₅ or O;
each Y is independently selected from the group consisting of a bond, methylene, aryl and heteroaryl; each R₅ is independently selected from the group consisting of H, optionally substituted C₁-C₆ alkyl, optionally substituted C₃-C₇ heterocycloalkyl and optionally substituted C₃-C₇ cycloalkyl; each k is independently 0, 1, 2, or 3; and m is 1, 2, 3, or 4. In some embodiments, n is 2; each R₃ is H; each R₁ and R₂ is independently selected from the group consisting of H, C₁-C₆ alkyl, and C₃-C₄ cycloalkyl.

In some embodiments, L is

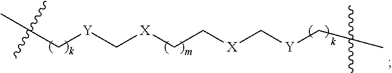

each X is independently NR₅ or 0;
each Y is aryl or heteroaryl; each R₅ is independently selected from the group consisting of H, optionally substituted C₁-C₆ alkyl, optionally substituted C₃-C₇ heterocycloalkyl and optionally substituted C₃-C₇ cycloalkyl; each k is 1; and m is 1, 2, 3, or 4. In some embodiments, n is 2; each R₃ is H; each R₁ and R₂ is independently selected from the group consisting of H, C₁-C₆ alkyl, and C₃-C₄ cycloalkyl.

In some embodiments, L is

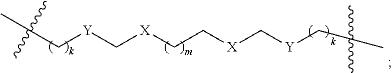

each X is independently NR₅ or O;
each Y is phenyl; each R₅ is independently selected from the group consisting of H, optionally substituted C₁-C₆ alkyl, optionally substituted C₃-C₇ heterocycloalkyl and optionally substituted C₃-C₇ cycloalkyl; each k is 1; and m is 1, 2, 3, or 4. In some embodiments, n is 2; each R₃ is H; each R₁ and R₂ is independently selected from the group consisting of H, C₁-C₆ alkyl, and C₃-C₄ cycloalkyl.

In some embodiments, L is

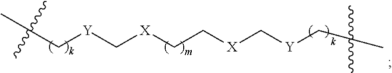

each X is independently O; each Y is aryl or heteroaryl; each k is 1; and m is 1, 2, 3, or 4. In some embodiments, n is 2; each R₃ is H; each R₁ and R₂ is independently selected from the group consisting of H, C₁-C₆ alkyl, and C₃-C₄ cycloalkyl.

In some embodiments, L is

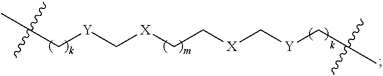

each X is independently O; each Y is phenyl; each k is 1; and m is 1, 2, 3, or 4. In some embodiments, n is 2; each R₃ is H; each R₁ and R₂ is independently selected from the group consisting of H, C₁-C₆ alkyl, and C₃-C₄ cycloalkyl.

In some embodiments, L is

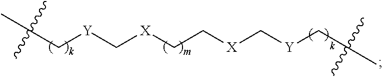

each X is independently NR₅; each Y is aryl or heteroaryl; each R₅ is independently selected from the group consisting of H, optionally substituted C₁-C₆ alkyl, optionally substituted C₃-C₇ heterocycloalkyl and optionally substituted C₃-C₇ cycloalkyl; each k is 1; and m is 1, 2, 3, or 4. In some embodiments, n is 2; each R₃ is H; each R₁ and R₂ is independently selected from the group consisting of H, C₁-C₆ alkyl, and C₃-C₄ cycloalkyl.

In some embodiments, L is

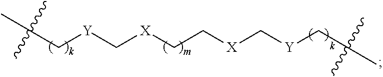

each X is independently NR₅; each Y is phenyl; each R₅ is independently selected from the group consisting of H, optionally substituted C₁-C₆ alkyl, optionally substituted C₃-C₇ heterocycloalkyl and optionally substituted C₃-C₇ cycloalkyl; each k is 1; and m is 1, 2, 3, or 4. In some embodiments, n is 2; each R₃ is H; each R₁ and R₂ is independently selected from the group consisting of H, C₁-C₆ alkyl, and C₃-C₄ cycloalkyl. In some embodiments, each R₅ is H.

In some embodiments, L is

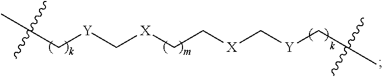

each X is independently NR₅ or O; each Y is independently selected from the group consisting of a bond, methylene; each R₅ is independently selected from the group consisting of H, optionally substituted C₁-C₆ alkyl, optionally substituted C₃-C₇ heterocycloalkyl and optionally substituted C₃-C₇ cycloalkyl; each k is independently 0, 1, 2, or 3; and m is 1, 2, 3, or 4. In some embodiments, n is 2; each R₃ is H; each R₁ and R₂ is independently selected from the group consisting of H, C₁-C₆ alkyl, and C₃-C₄ cycloalkyl.

In some embodiments, L is

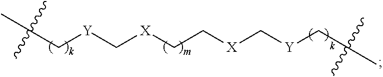

each X is NH; each Y is independently selected from the group consisting of a bond, methylene; each R₅ is independently selected from the group consisting of H, optionally substituted C₁-C₆ alkyl, optionally substituted C₃-C₇ heterocycloalkyl and optionally substituted C₃-C₇ cycloalkyl; each k is independently 1 or 2; and m is 1, 2, 3, or 4. In some embodiments, n is 2; each R₃ is H; each R₁ and R₂ is independently selected from the group consisting of H, C₁-C₆ alkyl, and C₃-C₄ cycloalkyl.

In some embodiments, L is

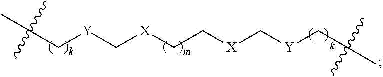

each X is O; each Y is independently selected from the group consisting of a bond, methylene; each R₅ is independently selected from the group consisting of H, optionally substituted C₁-C₆ alkyl, optionally substituted C₃-C₇ heterocycloalkyl and optionally substituted C₃-C₇ cycloalkyl; each k is independently 1 or 2; and m is 1, 2, 3, or 4. In some embodiments, n is 2; each R₃ is H; each R₁ and R₂ is independently selected from the group consisting of H, C₁-C₆ alkyl, and C₃-C₄ cycloalkyl.

In some embodiments, L is

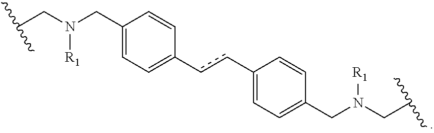

In some embodiments L is

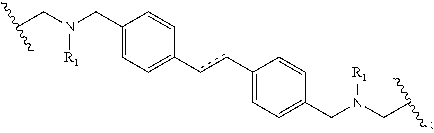

n is 2; each R₃ is independently H or —C(O)R₄; each R₁ and R₂ is independently selected from the group consisting of H, C₁-C₆ alkyl, and C₃-C₇ cycloalkyl. In some embodiments, n is 2; each R₃ is independently H or —C(O)R₄; each R₁ and R₂ is independently selected from the group consisting of H, C₁-C₆ alkyl, and C₃-C₄ cycloalkyl. In some embodiments, n is 2; each R₃ is H; each R₁ and R₂ is independently selected from the group consisting of H, C₁-C₆ alkyl, and C₃-C₄ cycloalkyl. In some embodiments, n is 2; each R₃ is H; each R₁ and R₂ is independently selected from the group consisting of H, methyl, ethyl, propyl, and cyclopropyl. In some embodiments, at least three R₁ are H. In some embodiments, at least two R₁ is H. In some embodiments, at least one R₁ is H. In some embodiments, each R₁ is H. In some embodiments, [symbol] is a single bond.

In some embodiments, L is

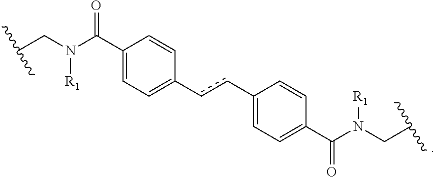

In some embodiments, L is

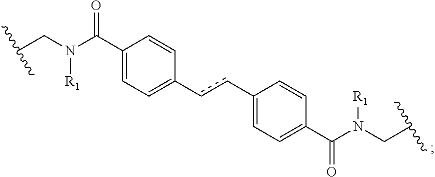

n is 2; each R₃ is independently H or —C(O)R₄; each R₁ and R₂ is independently selected from the group consisting of H, C₁-C₆ alkyl, and C₃-C₇ cycloalkyl. In some embodiments, n is 2; each R₃ is independently H or —C(O)R₄; each R₁ and R₂ is independently selected from the group consisting of H, C₁-C₆ alkyl, and C₃-C₄ cycloalkyl. In some embodiments, n is 2; each R₃ is H; each R₁ and R₂ is independently selected from the group consisting of H, C₁-C₆ alkyl, and C₃-C₄ cycloalkyl. In some embodiments, n is 2; each R₃ is H; each R₁ and R₂ is independently selected from the group consisting of H, methyl, ethyl, propyl, and cyclopropyl. In some embodiments, at least three R₁ are H. In some embodiments, at least two R₁ is H. In some embodiments, at least one R₁ is H. In some embodiments, each R₁ is H. In some embodiments, [symbol] is a single bond. In some embodiments, [symbol] is a double bond.

In some embodiments, L is

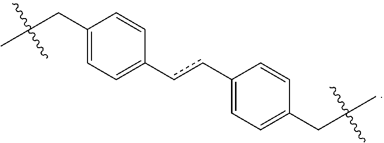

In some embodiments, L is

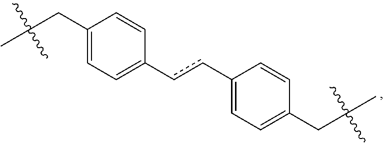

n is 2; each R3 is independently H or —C(O)R₄; each R₁ and R₂ is independently selected from the group consisting of H, C₁-C₆ alkyl, and C₃-C₇ cycloalkyl. In some embodiments, n is 2; each R₃ is independently H or —C(O)R₄; each R₁ and R₂ is independently selected from the group consisting of H, C₁-C₆ alkyl, and C₃-C₄ cycloalkyl. In some embodiments, n is 2; each R₃ is H; each R₁ and R₂ is independently selected from the group consisting of H, C₁-C₆ alkyl, and C₃-C₄ cycloalkyl. In some embodiments, n is 2; each R₃ is H; each R₁ and R₂ is independently selected from the group consisting of H, methyl, ethyl, propyl, and cyclopropyl. In some embodiments, at least three R₁ are H. In some embodiments, at least two R₁ is H. In some embodiments, at least one R₁ is H. In some embodiments, each R₁ is H. In some embodiments, [symbol] is a single bond. In some embodiments, [symbol] is a double bond.

In some embodiments, L is. In some embodiments, L is

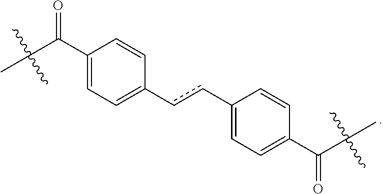

In some embodiments, L is

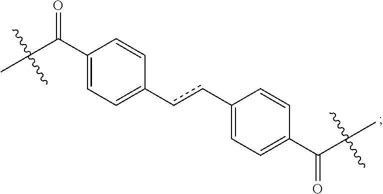

n is 2; each R3 is independently H or —C(O)R₄; each R₁ and R₂ is independently selected from the group consisting of H, C₁-C₆ alkyl, and C₃-C₇ cycloalkyl. In some embodiments, n is 2; each R₃ is independently H or —C(O)R₄; each R₁ and R₂ is independently selected from the group consisting of H, C₁-C₆ alkyl, and C₃-C₄ cycloalkyl. In some embodiments, n is 2; each R₃ is H; each R₁ and R₂ is independently selected from the group consisting of H, C₁-C₆ alkyl, and C₃-C₄ cycloalkyl. In some embodiments, n is 2; each R₃ is H; each R₁ and R₂ is independently selected from the group consisting of H, methyl, ethyl, propyl, and cyclopropyl. In some embodiments, at least three R₁ are H. In some embodiments, at least two R₁ is H. In some embodiments, at least one R₁ is H. In some embodiments, each R₁ is H. In some embodiments, [symbol] is a single bond. In some embodiments, [symbol] is a double bond.

In some embodiments, L is

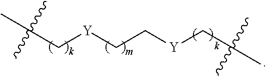

In some embodiments, L is

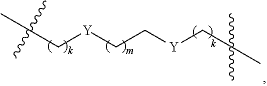

each Y is independently selected from the group consisting of a bond, methylene, aryl and heteroaryl; each k is independently 0, 1, 2, or 3; and m is 1, 2, 3, or 4; n is 2; each R3 is independently H or —C(O)R₄; each R₁ and R₂ is independently selected from the group consisting of H, C₁-C₆ alkyl, and C₃-C₇ cycloalkyl. In some embodiments, n is 2; each R₃ is independently H or —C(O)R₄; each R₁ and R₂ is independently selected from the group consisting of H, C₁-C₆ alkyl, and C₃-C₄ cycloalkyl. In some embodiments, n is 2; each R₃ is H; each R₁ and R₂ is independently selected from the group consisting of H, C₁-C₆ alkyl, and C₃-C₄ cycloalkyl. In some embodiments, n is 2; each R₃ is H; each R₁ and R₂ is independently selected from the group consisting of H, methyl, ethyl, propyl, and cyclopropyl. In some embodiments, at least three R₁ are H. In some embodiments, at least two R₁ is H. In some embodiments, at least one R₁ is H. In some embodiments, each R₁ is H. In some embodiments, [symbol] is a single bond. In some embodiments, [symbol] is a double bond.

In some embodiments, one or more compounds of Formula I are capable of inhibiting cellular proliferation. For example, In some embodiments, one or more compounds of Formula I inhibit proliferation of tumor cells or tumor cell lines. For example, such cell lines express a protein which is mutant. In some embodiments, the compounds of Formula I cell proliferation in vitro or in an in vivo model such as a xenograft mouse model. In some embodiments, in vitro cultured cell proliferation may be inhibited with an IC₅₀ of less than 100 μM, 75 μM, 50 μM, 25 μM, 15 μM, 10 μM, 5 μM, 3 μM, 2 μM, 1 μM or less by one or more compounds of Formula I.

In some embodiments, the compound of Formula 1 is racemic. In some embodiments, the compound of Formula 1 is meso. In some embodiments, the compound of Formula 1 is enantioenriched with an enantiomeric excess of greater than 99.9, 99.5, 99, 98, 97, 96, 95, 94, 93, 92, 91, 90, 88, 86, 84, 82, 80, 75, 70, 65, 60, 55, 50, 40, 30, 20, 10% ee. In some embodiments, the compound of Formula 1 is enantioenriched with an enantiomeric excess of greater than 95% ee. In some embodiments of the compounds disclosed herein, the compound of Formula 1 is a compound having the structure of Formula 1a:

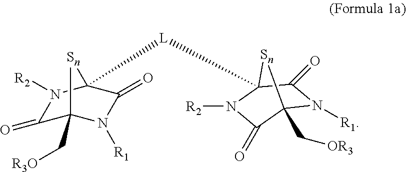

In some embodiments of the compounds disclosed herein, the compound of Formula 1 is a compound having the structure of Formula 1b:

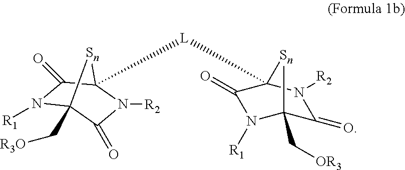

In some embodiments of the compounds disclosed herein, the compound of Formula 1 is a compound having the structure of Formula 1c:

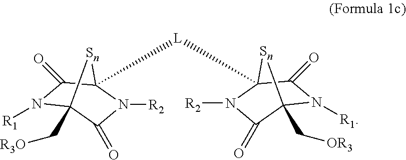

In some embodiments, the compound is a mixture of at least two compounds selected from the group consisting of Formula 1a, Formula 1b, and Formula 1c. In some embodiments, the compound is a mixture of Formula 1a and Formula 1b.
In some embodiments, the compound is a compound Table 1 or stereoisomer thereof.

TABLE 1
Exemplary Compounds of Formula 1

| Cmpd | | Calc | Measured |
|---|---|---|---|
| Number | Compound Structure | Mass | Mass |
| 1 | 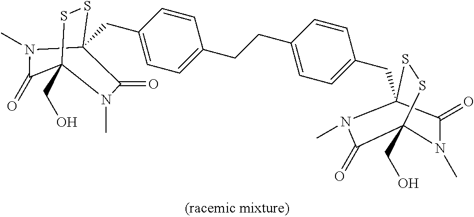 [SMILES: CN1C(=O)[C@]2(Cc3ccc(CCc4ccc(C[C@@]56SS[C@@](CO)(C(=O)N5C)N(C)C6=O)cc4)cc3)SS[C@@]1(CO)C(=O)N2C] | 674.1 | 674.87 (M + H) |
| 2 | 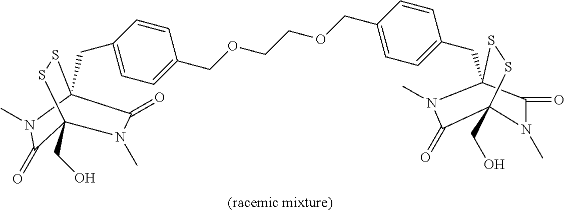 [SMILES: CN1C(=O)[C@]2(Cc3ccc(COCCOCc4ccc(C[C@@]56SS[C@@](CO)(C(=O)N5C)N(C)C6=O)cc4)cc3)SS[C@@]1(CO)C(=O)N2C] | 734.2 | 733.1 (M − H) |
| 3 | 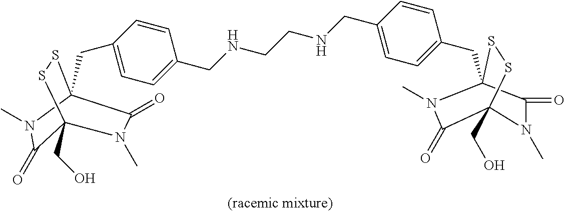 [SMILES: CN1C(=O)[C@]2(Cc3ccc(CNCCNCc4ccc(C[C@@]56SS[C@@](CO)(C(=O)N5C)N(C)C6=O)cc4)cc3)SS[C@@]1(CO)C(=O)N2C] | 732.2 | |
| 4 | 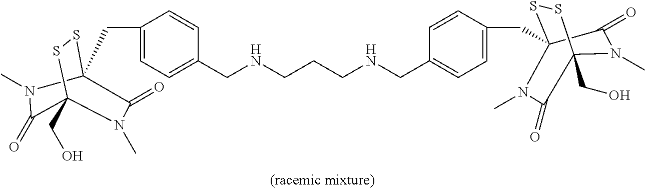 | 746.2 | |
| 5 | 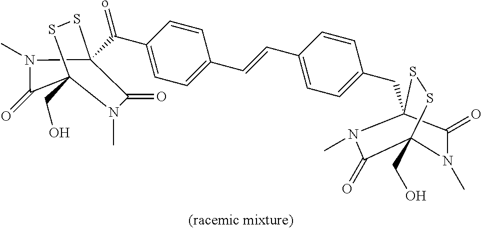 | 700.1 | |
| 6 | 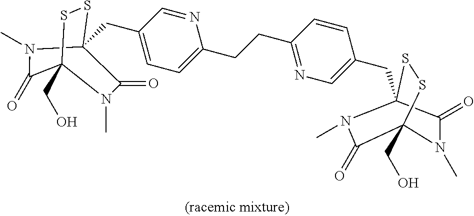 [SMILES: CN1C(=O)[C@]2(Cc3ccc(CCc4ccc(C[C@@]56SS[C@@](CO)(C(=O)N5C)N(C)C6=O)cn4)nc3)SS[C@@]1(CO)C(=O)N2C] | 676.1 | 677.9 (M + H) |

### B. Methods of Making

Compounds disclosed herein may be prepared by the routes described below. Materials used herein are either commercially available or prepared by synthetic methods generally known in the art. These schemes are not limited to the compounds listed or by any particular substituents, which are employed for illustrative purposes. Although various steps of are described and depicted in Scheme A, the steps in some cases may be performed in a different order than the order shown in Scheme A. Various modifications to these synthetic reaction schemes may be made and will be suggested to one skilled in the art having referred to the disclosure contained in this Application. Numbering does not necessarily correspond to that of claims or other tables.

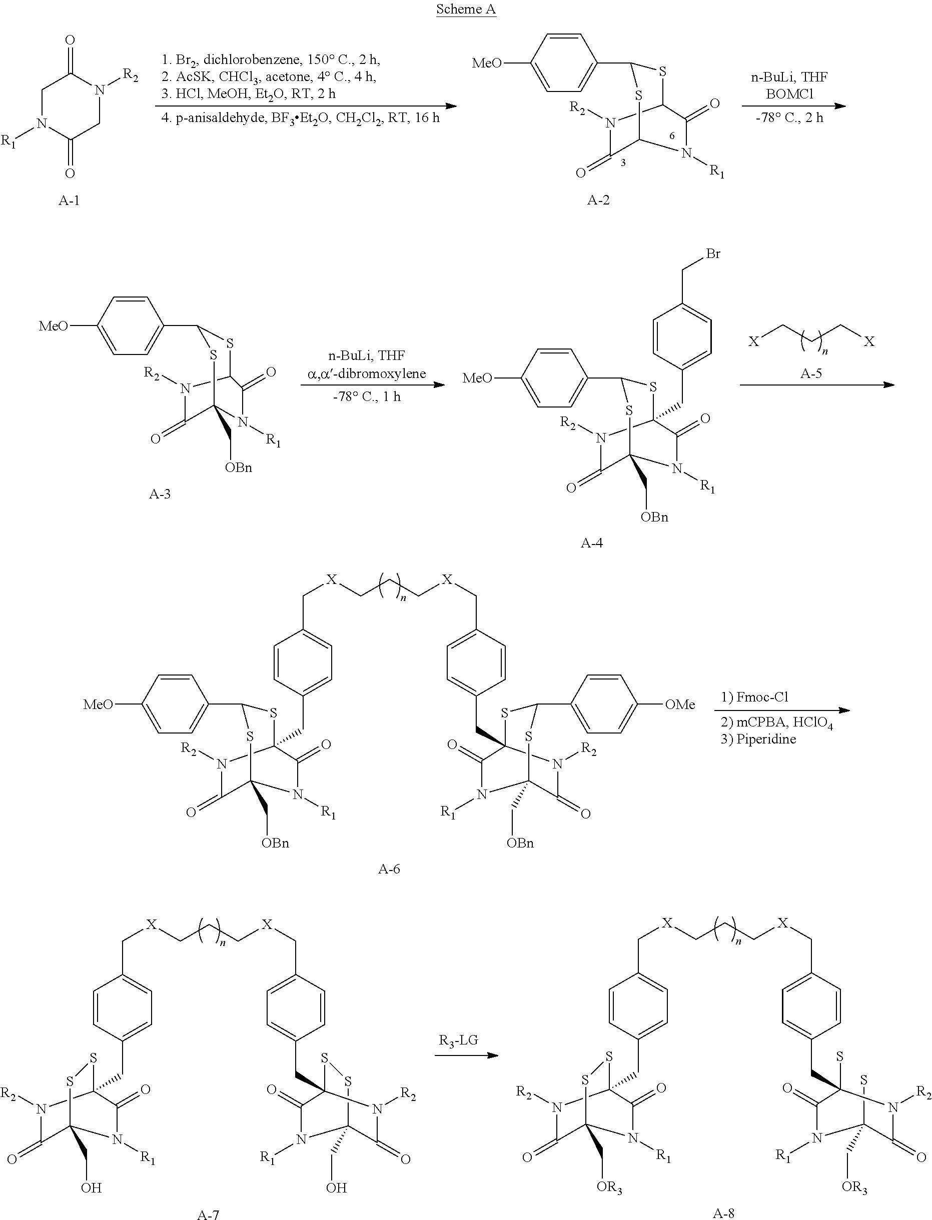

In Scheme A, A-1 is reacted with bromine. The dibrominated compound is then reacted with AcSK followed by acid alcoholysis and then reaction with an aldehyde in the presence of a Lewis acid to form the dithiane bicyclic compound A-2. Alkylation of A-2 is achieved with a lithium base such as butyl lithium and treatment with an alkylating agent such as benzyl chloromethyl ether to yield ether A-3. Subsequent alkylylation of A-3 is similarly carried out with a base such as butyl lithium and an dibromxylene to furnish A-4. Two equivalents of A-4 are attached to a suitable linker such as A-5 in th presence of base. Within A-5, each X can independently be OH or —NHR₄, and N can be 1, 2, 3, or 4. Dimerized compound A-6 can be deprotected to f orm bis alcohol A-7. One or both alcohols can then be reacted with an R₃ group such as an acyl or alkyl group as described in the compounds of Formula 1 via reaction with R₃-LG wherein LG is a leaving group in the presence of base.

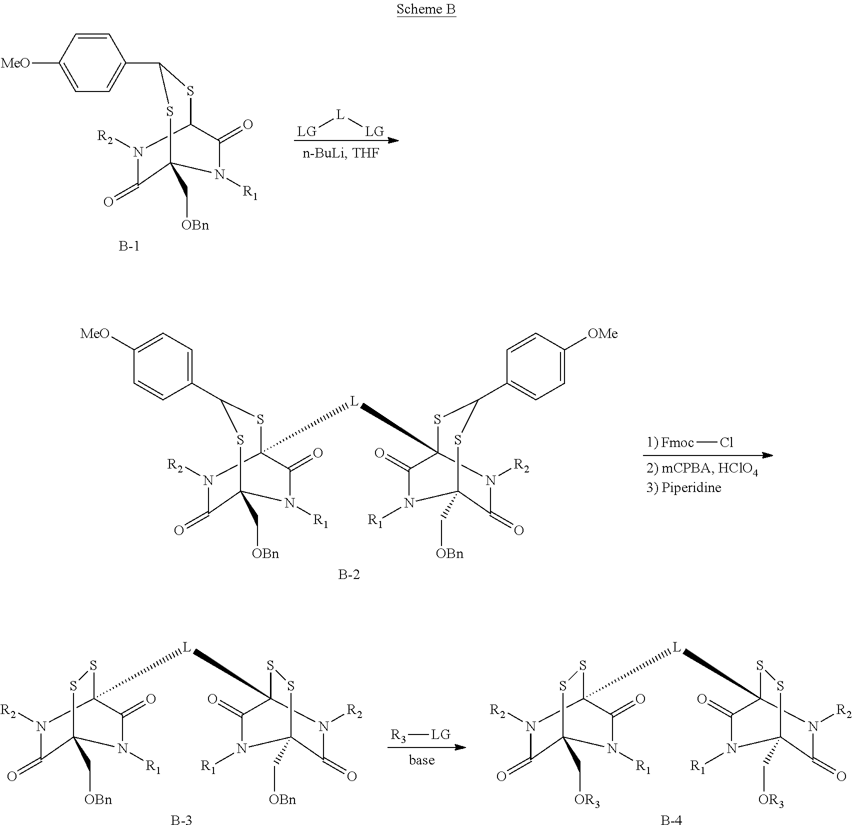

In scheme B, two equivalents of B-1 is reacted with a linker group comprising two leaving groups (LG) in the presence of base. The dimerized product, B-2 is deprotected to furnish B-3. The diol can be reacted with at least one R₃-LG complex to furnish compounds B-4.

### C. Pharmaceutical Compositions and Formulations

In some embodiments, the compounds described herein are formulated into pharmaceutical compositions. In specific embodiments, pharmaceutical compositions are formulated in a conventional manner using one or more physiologically acceptable carriers comprising excipients and auxiliaries which facilitate processing of the active compounds into preparations which can be used pharmaceutically. Proper formulation is dependent upon the route of administration chosen. Any pharmaceutically acceptable techniques, carriers, and excipients are used as suitable to formulate the pharmaceutical compositions described herein: Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

Provided herein are pharmaceutical compositions comprising a compound of Formula I, and a pharmaceutically acceptable diluent(s), excipient(s), or carrier(s). In certain embodiments, the compounds described are administered as pharmaceutical compositions in which compounds of Formula I, are mixed with other active ingredients, as in combination therapy. Encompassed herein are all combinations of actives set forth in the combination therapies section below and throughout this disclosure. In specific embodiments, the pharmaceutical compositions include one or more compounds of Formula I.

A pharmaceutical composition, as used herein, refers to a mixture of a compound of Formula I, with other chemical components, such as carriers, stabilizers, diluents, dispersing agents, suspending agents, thickening agents, and/or excipients. In certain embodiments, the pharmaceutical composition facilitates administration of the compound to an organism. In some embodiments, practicing the methods of treatment or use provided herein, therapeutically effective amounts of compounds of Formula I, provided herein are administered in a pharmaceutical composition to a mammal having a disease or condition to be treated. In specific embodiments, the mammal is a human. In certain embodiments, therapeutically effective amounts vary depending on the severity of the disease, the age and relative health of the subject, the potency of the compound used and other factors. The compounds described herein are used singly or in combination with one or more therapeutic agents as components of mixtures.

In one embodiment, one or more compounds of Formula I, is formulated in an aqueous solution. In specific embodiments, the aqueous solution is selected from, by way of example only, a physiologically compatible buffer, such as Hank's solution, Ringer's solution, or physiological saline buffer. In other embodiments, one or more compound of Formula I, is formulated for transmucosal administration. In specific embodiments, transmucosal formulations include penetrants that are appropriate to the barrier to be permeated. In still other embodiments wherein the compounds described herein are formulated for other parenteral injections, appropriate formulations include aqueous or nonaqueous solutions. In specific embodiments, such solutions include physiologically compatible buffers and/or excipients.

In another embodiment, compounds described herein are formulated for oral administration. Compounds described herein, including compounds of Formula I, are formulated by combining the active compounds with, e.g., pharmaceutically acceptable carriers or excipients. In various embodiments, the compounds described herein are formulated in oral dosage forms that include, by way of example only, tablets, powders, pills, dragees, capsules, liquids, gels, syrups, elixirs, slurries, suspensions and the like.

In certain embodiments, pharmaceutical preparations for oral use are obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as: for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. In specific embodiments, disintegrating agents are optionally added. Disintegrating agents include, by way of example only, cross-linked croscarmellose sodium, polyvinylpyrrolidone, agar, or alginic acid or a salt thereof such as sodium alginate.

In one embodiment, dosage forms, such as dragee cores and tablets, are provided with one or more suitable coating. In specific embodiments, concentrated sugar solutions are used for coating the dosage form. The sugar solutions, optionally contain additional components, such as by way of example only, gum arabic, talc, polyvinylpyrrolidone, carbopol gel, polyethylene glycol, and/or titanium dioxide, lacquer solutions, and suitable organic solvents or solvent mixtures. Dyestuffs and/or pigments are also optionally added to the coatings for identification purposes. Additionally, the dyestuffs and/or pigments are optionally utilized to characterize different combinations of active compound doses.

In certain embodiments, therapeutically effective amounts of at least one of the compounds described herein are formulated into other oral dosage forms. Oral dosage forms include push-fit capsules made of gelatin, as well as soft, sealed capsules made of gelatin and a plasticizer, such as glycerol or sorbitol. In specific embodiments, push-fit capsules contain the active ingredients in admixture with one or more filler. Fillers include, by way of example only, lactose, binders such as starches, and/or lubricants such as talc or magnesium stearate and, optionally, stabilizers. In other embodiments, soft capsules, contain one or more active compound that is dissolved or suspended in a suitable liquid. Suitable liquids include, by way of example only, one or more fatty oil, liquid paraffin, or liquid polyethylene glycol. In addition, stabilizers are optionally added.

In other embodiments, therapeutically effective amounts of at least one of the compounds described herein are formulated for buccal or sublingual administration. Formulations suitable for buccal or sublingual administration include, by way of example only, tablets, lozenges, or gels. In still other embodiments, the compounds described herein are formulated for parental injection, including formulations suitable for bolus injection or continuous infusion. In specific embodiments, formulations for injection are presented in unit dosage form (e.g., in ampoules) or in multi-dose containers. Preservatives are, optionally, added to the injection formulations. In still other embodiments, the pharmaceutical composition of a compound of Formula I is formulated in a form suitable for parenteral injection as sterile suspension, solution or emulsion in oily or aqueous vehicles. Parenteral injection formulations optionally contain formulatory agents such as suspending, stabilizing and/or dispersing agents. In specific embodiments, pharmaceutical formulations for parenteral administration include aqueous solutions of the active compounds in water-soluble form. In additional embodiments, suspensions of the active compounds are prepared as appropriate oily injection suspensions. Suitable lipophilic solvents or vehicles for use in the pharmaceutical compositions described herein include, by way of example only, fatty oils such as sesame oil, or synthetic fatty acid esters, such as ethyl oleate or triglycerides, or liposomes. In certain specific embodiments, aqueous injection suspensions contain substances which increase the viscosity of the suspension, such as sodium carboxymethyl cellulose, sorbitol, or dextran. Optionally, the suspension contains suitable stabilizers or agents which increase the solubility of the compounds to allow for the preparation of highly concentrated solutions. Alternatively, in other embodiments, the active ingredient is in powder form for constitution with a suitable vehicle, e.g., sterile pyrogen-free water, before use.

In still other embodiments, the compounds of Formula I are administered topically. The compounds described herein are formulated into a variety of topically administrable compositions, such as solutions, suspensions, lotions, gels, pastes, medicated sticks, balms, creams or ointments. Such pharmaceutical compositions optionally contain solubilizers, stabilizers, tonicity enhancing agents, buffers and preservatives.

In yet other embodiments, the compounds of Formula I are formulated for transdermal administration. In specific embodiments, transdermal formulations employ transdermal delivery devices and transdermal delivery patches and can be lipophilic emulsions or buffered, aqueous solutions, dissolved and/or dispersed in a polymer or an adhesive. In various embodiments, such patches are constructed for continuous, pulsatile, or on demand delivery of pharmaceutical agents. In additional embodiments, the transdermal delivery of the compounds of Formula I, is accomplished by means of iontophoretic patches and the like. In certain embodiments, transdermal patches provide controlled delivery of the compounds of Formula I. In specific embodiments, the rate of absorption is slowed by using rate-controlling membranes or by trapping the compound within a polymer matrix or gel. In alternative embodiments, absorption enhancers are used to increase absorption. Absorption enhancers or carriers include absorbable pharmaceutically acceptable solvents that assist passage through the skin. For example, in one embodiment, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin.

In other embodiments, the compounds of Formula I, are formulated for administration by inhalation. Various forms suitable for administration by inhalation include, but are not limited to, aerosols, mists or powders. Pharmaceutical compositions of Formula I, are conveniently delivered in the form of an aerosol spray presentation from pressurized packs or a nebuliser, with the use of a suitable propellant (e.g., dichlorodifluoromethane, trichlorofluoromethane, dichlorotetrafluoroethane, carbon dioxide or other suitable gas). In specific embodiments, the dosage unit of a pressurized aerosol is determined by providing a valve to deliver a metered amount. In certain embodiments, capsules and cartridges of, such as, by way of example only, gelatin for use in an inhaler or insufflator are formulated containing a powder mix of the compound and a suitable powder base such as lactose or starch.

In still other embodiments, the compounds of Formula I, are formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as, but not limited to, a mixture of fatty acid glycerides, optionally in combination with cocoa butter is first melted.

In certain embodiments, pharmaceutical compositions are formulated in any conventional manner using one or more physiologically acceptable carriers comprising excipients and auxiliaries which facilitate processing of the active compounds into preparations which can be used pharmaceutically. Proper formulation is dependent upon the route of administration chosen. Any pharmaceutically acceptable techniques, carriers, and excipients are optionally used as suitable. Pharmaceutical compositions comprising a compound of Formula I, are manufactured in a conventional manner, such as, by way of example only, by means of conventional mixing, dissolving, granulating, dragee-making, levigating, emulsifying, encapsulating, entrapping or compression processes.

Pharmaceutical compositions include at least one pharmaceutically acceptable carrier, diluent or excipient and at least one compound of Formula I, described herein as an active ingredient. The active ingredient is in free-acid or free-base form, or in a pharmaceutically acceptable salt form. In addition, the methods and pharmaceutical compositions described herein include the use of N-oxides, crystalline forms (also known as polymorphs), as well as active metabolites of these compounds having the same type of activity. All tautomers of the compounds described herein are included within the scope of the compounds presented herein. Additionally, the compounds described herein encompass unsolvated as well as solvated forms with pharmaceutically acceptable solvents such as water, ethanol, and the like. The solvated forms of the compounds presented herein are also considered to be disclosed herein. In addition, the pharmaceutical compositions optionally include other medicinal or pharmaceutical agents, carriers, adjuvants, such as preserving, stabilizing, wetting or emulsifying agents, solution promoters, salts for regulating the osmotic pressure, buffers, and/or other therapeutically valuable substances.

Methods for the preparation of compositions comprising the compounds described herein include formulating the compounds with one or more inert, pharmaceutically acceptable excipients or carriers to form a solid, semi-solid or liquid. Solid compositions include, but are not limited to, powders, tablets, dispersible granules, capsules, cachets, and suppositories. Liquid compositions include solutions in which a compound is dissolved, emulsions comprising a compound, or a solution containing liposomes, micelles, or nanoparticles comprising a compound as disclosed herein. Semi-solid compositions include, but are not limited to, gels, suspensions and creams. The form of the pharmaceutical compositions described herein include liquid solutions or suspensions, solid forms suitable for solution or suspension in a liquid prior to use, or as emulsions. These compositions also optionally contain minor amounts of nontoxic, auxiliary substances, such as wetting or emulsifying agents, pH buffering agents, and so forth.

In some embodiments, a pharmaceutical composition comprising at least one compound of Formula I, illustratively takes the form of a liquid where the agents are present in solution, in suspension or both. Typically when the composition is administered as a solution or suspension a first portion of the agent is present in solution and a second portion of the agent is present in particulate form, in suspension in a liquid matrix. In some embodiments, a liquid composition includes a gel formulation. In other embodiments, the liquid composition is aqueous.

In certain embodiments, useful aqueous suspension contain one or more polymers as suspending agents. Useful polymers include water-soluble polymers such as cellulosic polymers, e.g., hydroxypropyl methylcellulose, and water-insoluble polymers such as cross-linked carboxyl-containing polymers. Certain pharmaceutical compositions described herein comprise a mucoadhesive polymer, selected for example from carboxymethylcellulose, carbomer (acrylic acid polymer), poly(methylmethacrylate), polyacrylamide, polycarbophil, acrylic acid/butyl acrylate copolymer, sodium alginate and dextran.

Useful pharmaceutical compositions also, optionally, include solubilizing agents to aid in the solubility of a compound of Formula I. The term “solubilizing agent” generally includes agents that result in formation of a micellar solution or a true solution of the agent. Certain acceptable nonionic surfactants, for example polysorbate 80, are useful as solubilizing agents, as can ophthalmically acceptable glycols, polyglycols, e.g., polyethylene glycol 400, and glycol ethers.

Furthermore, useful pharmaceutical compositions optionally include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Additionally, useful compositions also, optionally, include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Other useful pharmaceutical compositions optionally include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

Still other useful compositions include one or more surfactants to enhance physical stability or for other purposes. Suitable nonionic surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

Still other useful compositions include one or more antioxidants to enhance chemical stability where required. Suitable antioxidants include, by way of example only, ascorbic acid and sodium metabisulfite.

In certain embodiments, aqueous suspension compositions are packaged in single-dose non-reclosable containers. Alternatively, multiple-dose reclosable containers are used, in which case it is typical to include a preservative in the composition.

In alternative embodiments, other delivery systems for hydrophobic pharmaceutical compounds are employed. Liposomes and emulsions are examples of delivery vehicles or carriers useful herein. In certain embodiments, organic solvents such as N-methylpyrrolidone are also employed. In additional embodiments, the compounds described herein are delivered using a sustained-release system, such as semipermeable matrices of solid hydrophobic polymers containing the therapeutic agent. Various sustained-release materials are useful herein. In some embodiments, sustained-release capsules release the compounds for a few weeks up to over 100 days. Depending on the chemical nature and the biological stability of the therapeutic reagent, additional strategies for protein stabilization are employed.

In certain embodiments, the formulations described herein comprise one or more antioxidants, metal chelating agents, thiol containing compounds and/or other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (l) pentosan polysulfate and other heparinoids, (m) divalent cations such as magnesium and zinc; or (n) combinations thereof.

In some embodiments, a formulations described herein comprise N,N-Dimethylacetamide, Hydroxypropyl beta-Cyclodextrin, and water. For example, the formulations can comprise about 5% (v/v) N,N-Dimethylacetamide, about 50% (v/v) of 60% (w/v) Hydroxypropyl beta-Cyclodextrin, and about 45% (v/v) Sterile water. In some embodiments, a formulation comprising N,N-Dimethylacetamide, Hydroxypropyl beta-Cyclodextrin, and water is used for injection by intravenous route.

In some embodiments, a formulations described herein comprise N,N-Dimethylacetamide, Sulfobutyl ether beta-cyclodextrin (SBEβCD) and Sterile water. For example, the formulations can comprise about 5% (v/v) N,N-Dimethylacetamide, about 50% (v/v) of 60% (w/v) Sulfobutyl ether beta-cyclodextrin (SBEβCD) and about 45% (v/v) Sterile water. In some embodiments, a formulation comprising is used for consumption by an oral route.

### D. Routes of Administration

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In certain embodiments, a compound as described herein is administered in a local rather than systemic manner, for example, via injection of the compound directly into an organ, often in a depot preparation or sustained release formulation. In specific embodiments, long acting formulations are administered by implantation (for example subcutaneously or intramuscularly) or by intramuscular injection. Furthermore, in other embodiments, the drug is delivered in a targeted drug delivery system, for example, in a liposome coated with organ-specific antibody. In such embodiments, the liposomes are targeted to and taken up selectively by the organ. In yet other embodiments, the compound as described herein is provided in the form of a rapid release formulation, in the form of an extended release formulation, or in the form of an intermediate release formulation. In yet other embodiments, the compound described herein is administered topically.

### E. Kits/Articles of Manufacture

For use in the therapeutic applications described herein, kits and articles of manufacture are also provided. In some embodiments, such kits comprise a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. The containers are formed from a variety of materials such as glass or plastic.

The articles of manufacture provided herein contain packaging materials. Packaging materials for use in packaging pharmaceutical products Include those found in, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. For example, the container(s) includes one or more compounds described herein, optionally in a composition or in combination with another agent as disclosed herein. The container(s) optionally have a sterile access port (for example the container is an intravenous solution bag or a vial having a stopper pierceable by a hypodermic injection needle). Such kits optionally comprising a compound with an identifying description or label or instructions relating to its use in the methods described herein.

For example, a kit typically includes one or more additional containers, each with one or more of various materials (such as reagents, optionally in concentrated form, and/or devices) desirable from a commercial and user standpoint for use of a compound described herein. Non-limiting examples of such materials include, but not limited to, buffers, diluents, filters, needles, syringes; carrier, package, container, vial and/or tube labels listing contents and/or instructions for use, and package inserts with instructions for use. A set of instructions will also typically be included. A label is optionally on or associated with the container. For example, a label is on a container when letters, numbers or other characters forming the label are attached, molded or etched into the container itself, a label is associated with a container when it is present within a receptacle or carrier that also holds the container, e.g., as a package insert. In addition, a label is used to indicate that the contents are to be used for a specific therapeutic application. In addition, the label indicates directions for use of the contents, such as in the methods described herein. In certain embodiments, the pharmaceutical compositions is presented in a pack or dispenser device which contains one or more unit dosage forms containing a compound provided herein. The pack for example contains metal or plastic foil, such as a blister pack. Or, the pack or dispenser device is accompanied by instructions for administration. Or, the pack or dispenser is accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In some embodiments, compositions containing a compound provided herein formulated in a compatible pharmaceutical carrier are prepared, placed in an appropriate container, and labeled for treatment of an indicated condition.

### F. Methods of Use

In some embodiments, the chemical entities described herein are used for the treatment of cancers of the
i. digestive system including, without limitation, the esophagus, stomach, small intestine, colon (including colorectal), liver & intrahepatic bile duct, gallbladder & other biliary, pancreas, spleen and other digestive organs;
ii. respiratory system, including without limitation, larynx, lung & bronchus, and other respiratory organs;
iii. skin;
iv. thyroid;
v. breast;
vi. genital system, including without limitation, uterine cervix, ovary, and prostate;
vii. urinary system, including without limitation, urinary bladder and kidney and renal pelvis;
viii. oral cavity & pharynx, including without limitation, tongue, mouth, pharynx, and other oral cavity;
ix. Brain, astrocytomas, GBM, medullablastoma, and other brain tumors;
x. Sarcoma including soft and bone related tumors; and
xi. viral infections and cancers related to viral infections.

In some embodiments, the chemical entities described herein are used for the treatment of colon cancer, liver cancer, lung cancer, melanoma, thyroid cancer, breast cancer, ovarian cancer, and oral cancer.

The chemical entities described herein may also be used in conjunction with other well known therapeutic agents that are selected for their particular usefulness against the condition that is being treated. For example, the chemical entities described herein may be useful in combination with at least one additional anti-cancer and/or cytotoxic agents. Further, the chemical entities described herein may also be useful in combination with other inhibitors of parts of the signaling pathway that links cell surface growth factor receptors to nuclear signals initiating cellular proliferation.

Such known anti-cancer and/or cytotoxic agents that may be used in combination with the chemical entities described herein include:

(i) other antiproliferative/antineoplastic drugs and combinations thereof, as used in medical oncology, such as alkylating agents (for example cis-platin, oxaliplatin, carboplatin, cyclophosphamide, nitrogen mustard, melphalan, chlorambucil, busulphan, temozolamide and nitrosoureas); antimetabolites (for example gemcitabine and antifolates such as fluoropyrimidines like 5-fluorouracil and tegafur, raltitrexed, methotrexate, cytosine arabinoside, and hydroxyurea); antitumor antibiotics (for example anthracyclines like adriamycin, bleomycin, doxorubicin, daunomycin, epirubicin, idarubicin, mitomycinC, dactinomycin and mithramycin); antimitotic agents (for example vinca alkaloids like vincristine, vinblastine, vindesine and vinorelbine and taxoids like taxol and taxotere and polokinase inhibitors); and topoisomerase inhibitors (for example epipodophyllotoxins like etoposide and teniposide, amsacrine, topotecan and camptothecin);

(ii) cytostatic agents such as antioestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5a-reductase such as finasteride;

(iii) anti-invasion agents [for example c-Src kinase family inhibitors like 4-(6-chloro-2,3methylenedioxyanilino)-7-[2-(4-methylpiperazin-1-yl)ethoxy]-5-tetrahydropyran-4yloxyquinazoline (AZD0530; International Patent Application WO 01/94341), N-(2-chloro-6-methylphenyl)-2-{6-[4-(2-hydroxyethyl)piperazin-1-yl]-2-methylpyrimidin-4ylamino}thiazole-5-carboxamide (dasatinib, BMS-354825; J. Med. Chem., 2004, 47, 66586661) and bosutinib (SK1-606), and metalloproteinase inhibitors like marimastat, inhibitors of urokinase plasminogen activator receptor function or antibodies to Heparanase];

(iv) inhibitors of growth factor function: for example such inhibitors include growth factor antibodies and growth factor receptor antibodies (for example the anti-erbB2 antibody trastuzumab [Herceptin™], the anti-EGFR antibody panitumumab, the anti-erbB 1 antibody cetuximab [Erbitux, C225] and any growth factor or growth factor receptor antibodies disclosed by Stem et al. Critical reviews in oncology/haematology, 2005, Vol. 54, pp 11-29); such inhibitors also include tyrosine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, ZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)-quinazolin-4-amine (CI 1033), erbB2 tyrosine kinase inhibitors such as lapatinib); inhibitors of the hepatocyte growth factor family; inhibitors of the insulin growth factor family; inhibitors of the platelet-derived growth factor family such as imatinib and/or nilotinib (AMN107); inhibitors of serine/threonine kinases (for example Ras/Raf signalling inhibitors such as farnesyl transferase inhibitors, for example sorafenib (BAY 43-9006), tipifarnib (RI15777) and lonafarnib (SCH66336)), inhibitors of cell signalling through MEK and/or AKT kinases, c-kit inhibitors, abl kinase inhibitors, P13 kinase inhibitors, P1t3 kinase inhibitors, CSF-IR kinase inhibitors, IGF receptor (insulin like growth factor) kinase inhibitors; aurora kinase inhibitors (for example AZD1152, PH739358, VX-680, MLN8054, R763, MP235, MP529, VX-528 and AX39459) and cyclin dependent kinase inhibitors such as CDK2 and/or CDK4 inhibitors;

(v) antiangiogenic agents such as those which inhibit the effects of vascular endothelial growth factor, [for example the anti-vascular endothelial cell growth factor antibody bevacizumab (Avastin™) and for example, a VEGF receptor tyrosine kinase inhibitor such as vandetanib(ZD6474), vatalanib (PTK787), sunitinib (SU11248), axitinib (AG-013736), pazopanib (GW 786034) and 4-{4-fluoro-2-methylindol-5-yloxy)-6-methoxy-7-(3pyrrolidin-1-ylpropoxy)quinazoline (AZD2171; Example 240 within WO 00/47212), compounds such as those disclosed in International Patent Applications WO 97/22596, WO 97/30035, WO 97/32856 and WO 98/13354 and compounds that work by other mechanisms (for example linomide, inhibitors of integrin av˜-3 function and angiostatin));

(vi) vascular damaging agents such as Combretastatin A4 and compounds disclosed in International Patent Applications WO 99/02166, WO 00/40529, WO 00/41669, WO 01/92224, WO 02/04434 and WO 02/08213;

(vii) an endothelin receptor antagonist, for example zibotentan (ZD4054) or atrasentan;
(viii) antisense therapies, for example those which are directed to the targets listed above, such as ISIS 2503, an anti-ras antisense;

(ix) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase subject tolerance to chemotherapy or radiotherapy such as multi-drug resistance gene therapy; and

(x) immunotherapy approaches, including for example ex-vivo and in-vivo approaches to increase the immunogenicity of subject's tumor cells, such as transfection with cytokines such as interleukin 2, interleukin 4 or granulocyte-macrophage colony stimulating factor, approaches to decrease T-cell energy, approaches using transfected immune cells such as cytokine-transfected dendritic cells, approaches using cytokine-transfected tumor cell lines and approaches using anti-idiotypic antibodies.

In certain embodiments, the at least one chemical entity is administered in combination with one or more agents chosen from pacliataxel, bortezomib, dacarbazine, gemcitabine, trastuzumab, bevacizumab, capecitabine, docetaxel, erlotinib, aromatase inhibitors, such as AROMASIN™ (exemestane), and estrogen receptor inhibitors, such as FASLODEX™ (fulvestrant).

When a chemical entity described herein is administered into a human subject, the daily dosage will normally be determined by the prescribing physician with the dosage generally varying according to the age, weight, and response of the individual subject, as well as the severity of the subject's symptoms.

In one exemplary application, a suitable amount of at least one chemical entity is administered to a mammal undergoing treatment for cancer, for example, breast cancer. Administration typically occurs in an amount of between about 0.01 mg/kg of body weight to about 100 mg/kg of body weight per day (administered in single or divided doses), such as at least about 0.1 mg/kg of body weight per day. A particular therapeutic dosage can include, e.g., from about 0.01 mg to about 1000 mg of the chemical entity, such as including, e.g., from about 1 mg to about 1000 mg. The quantity of the at least one chemical entity in a unit dose of preparation may be varied or adjusted from about 0.1 mg to 1000 mg, such as from about 1 mg to 300 mg, for example 10 mg to 200 mg, according to the particular application. The amount administered will vary depending on the particular IC₅₀ value of the at least one chemical entity used and the judgment of the attending clinician taking into consideration factors such as health, weight, and age. In combinational applications in which the at least one chemical entity described herein is not the sole active ingredient, it may be possible to administer lesser amounts of the at least one chemical entity and still have therapeutic or prophylactic effect.

In some embodiments, the pharmaceutical preparation is in unit dosage form. In such form, the preparation is subdivided into unit doses containing appropriate quantities of the active component, e.g., an effective amount to achieve the desired purpose.

The actual dosage employed may be varied depending upon the requirements of the subject and the severity of the condition being treated. Determination of the proper dosage for a particular situation is within the skill of the art. Generally, treatment is initiated with smaller dosages which are less than the optimum dose of the at least one chemical entity. Thereafter, the dosage is increased by small amounts until the optimum effect under the circumstances is reached. For convenience, the total daily dosage may be divided and administered in portions during the day if desired.

The amount and frequency of administration of the at least one chemical entities described herein, and if applicable other chemotherapeutic agents and/or radiation therapy, will be regulated according to the judgment of the attending clinician (physician) considering such factors as age, condition and size of the subject as well as severity of the disease being treated.

The chemotherapeutic agent and/or radiation therapy can be administered according to therapeutic protocols well known in the art. It will be apparent to those skilled in the art that the administration of the chemotherapeutic agent and/or radiation therapy can be varied depending on the disease being treated and the known effects of the chemotherapeutic agent and/or radiation therapy on that disease. Also, in accordance with the knowledge of the skilled clinician, the therapeutic protocols (e.g., dosage amounts and times of administration) can be varied in view of the observed effects of the administered therapeutic agents (i.e., antineoplastic agent or radiation) on the subject, and in view of the observed responses of the disease to the administered therapeutic agents.

Also, in general, the at least one chemical entities described herein need not be administered in the same pharmaceutical composition as a chemotherapeutic agent, and may, because of different physical and chemical characteristics, be administered by a different route. For example, the chemical entities/compositions may be administered orally to generate and maintain good blood levels thereof, while the chemotherapeutic agent may be administered intravenously. The determination of the mode of administration and the advisability of administration, where possible, in the same pharmaceutical composition, is well within the knowledge of the skilled clinician. The initial administration can be made according to established protocols known in the art, and then, based upon the observed effects, the dosage, modes of administration and times of administration can be modified by the skilled clinician.

The particular choice of chemical entity (and where appropriate, chemotherapeutic agent and/or radiation) will depend upon the diagnosis of the attending physicians and their judgment of the condition of the subject and the appropriate treatment protocol.

The chemical entities described herein (and where appropriate chemotherapeutic agent and/or radiation) may be administered concurrently (e.g., simultaneously, essentially simultaneously or within the same treatment protocol) or sequentially, depending upon the nature of the proliferative disease, the condition of the subject, and the actual choice of chemotherapeutic agent and/or radiation to be administered in conjunction (i.e., within a single treatment protocol) with the chemical entity/composition.

In combinational applications and uses, the chemical entity/composition and the chemotherapeutic agent and/or radiation need not be administered simultaneously or essentially simultaneously, and the initial order of administration of the chemical entity/composition, and the chemotherapeutic agent and/or radiation, may not be important. Thus, the at least one chemical entity described herein may be administered first followed by the administration of the chemotherapeutic agent and/or radiation; or the chemotherapeutic agent and/or radiation may be administered first followed by the administration of the at least one chemical entity described herein. This alternate administration may be repeated during a single treatment protocol. The determination of the order of administration, and the number of repetitions of administration of each therapeutic agent during a treatment protocol, is well within the knowledge of the skilled physician after evaluation of the disease being treated and the condition of the subject. For example, the chemotherapeutic agent and/or radiation may be administered first, and then the treatment continued with the administration of the at least one chemical entity described herein followed, where determined advantageous, by the administration of the chemotherapeutic agent and/or radiation, and so on until the treatment protocol is complete.

Thus, in accordance with experience and knowledge, the practicing physician can modify each protocol for the administration of a chemical entity/composition for treatment according to the individual subject's needs, as the treatment proceeds.

The attending clinician, in judging whether treatment is effective at the dosage administered, will consider the general well-being of the subject as well as more definite signs such as relief of disease-related symptoms, inhibition of tumor growth, actual shrinkage of the tumor, or inhibition of metastasis. Size of the tumor can be measured by standard methods such as radiological studies, e.g., CAT or MRI scan, and successive measurements can be used to judge whether or not growth of the tumor has been retarded or even reversed. Relief of disease-related symptoms such as pain, and improvement in overall condition can also be used to help judge effectiveness of treatment.

### Antiviral Therapy:

p300 is a key transcriptional co-activator that modulates many cellular processes including epigenetics and cell differentiation. It consists of multiple zinc finger domains referred to as cysteine-histidine region or CH domain (CH₁—CH₃) that are involved in protein-protein interactions with other transcriptional factors and enzymes. Such p300CH1 mediated protein-protein interactions (PPIs) are not limited to human cellular machinery but can also be found in virus-host interactions. Two examples of a such a binding interaction are the interaction between p300 and oncoproteins AD E1A, HPV16-E7. These interactions have been shown to play a significant role in affecting host immune response to defend against infection (AD E1A and STAT2 mediated) and ability to maintain apoptosis (HPV E6 and p53 mediated). In addition to these two viral oncoproteins interactions, p300 is also known to interact with Hepatitis C virus core protein and to affect transcriptional and acetylation activities of the host cell.

In summary, p300_{CH1} domain can be a target for anti-viral treatment as well as virus-mediated cancers such as cervical cancer. However, since these are PPIs developing small molecules that can effectively, disrupt them has been an enormous challenge.

The chemical entities described herein bind directly to p300_{CH1} domain and thereby can potentially disrupt p300 binding with viral proteins.

In some embodiments, the chemical entities described herein are used for the treatment of a viral infection. In some embodiments, the chemical entities described herein are used for the treatment of a viral infection such as an infection of human papilloma virus (HPV), hepatitis C (HCV), or adenovirus.

### EXAMPLES

The following examples serve to more fully describe the manner of using the invention. These examples are presented for illustrative purposes and should not serve to limit the true scope of the invention.

In carrying out the procedures of the methods described herein, it is of course to be understood that reference to particular buffers, media, reagents, cells, culture conditions and the like are not intended to be limiting, but are to be read so as to include all related materials that one of ordinary skill in the art would recognize as being of interest or value in the particular context in which that discussion is presented. For example, it is often possible to substitute one buffer system or culture medium for another and still achieve similar, if not identical, results. Those of skill in the art will have sufficient knowledge of such systems and methodologies so as to be able, without undue experimentation, to make such substitutions as will optimally serve their purposes in using the methods and procedures disclosed herein.

### Example 1

### Synthesis of 1,1′-ethane-1,2-diylbis[4-(iodomethyl)benzene] (I-8)

The compound 1,1′-ethane-1,2-diylbis[4-(iodomethyl)benzene] I-8 was synthesized according to the following scheme:

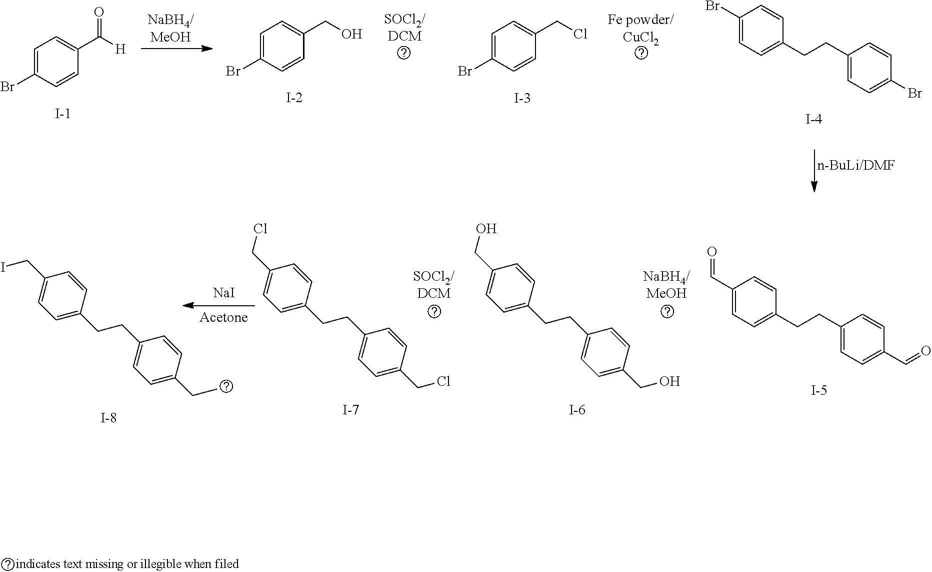

### Step-1: Preparation of (4-bromo-phenyl)-methanol (I-2)

To a solution of 4-bromo-benzaldehyde (20.0 g, 0.1080 mol) in methanol (200 mL) was added sodium borohydride (4.79 g, 0.1297 mol) at ice temperature. Resulting reaction mass was stirred at room temperature for 10 minutes. Reaction mass was quenched with saturated ammonium chloride solution (100 mL), aqueous layer was extracted with ethyl acetate (2×800 mL). Organic layer was washed with water (2×250 mL), dried over anhydrous sodium sulphate and filtered. The volatiles were concentrated under reduced pressure to obtain the product as off white solid (19.0 g, 94.0%).

### Step-2: Preparation of 4-bromo-4-chloromethyl benzene (I-3)

To a solution of (4-bromo-phenyl)-methanol (19.0 g, 0.1015 mol) in dichloromethane (190 mL) was added thionyl chloride (18.89 mL 0.2539 mol) slowly at ice temperature and catalytic DMAP. Resulting reaction mass was stirred at room temperature for 10 minutes. Reaction mass was concentrated, diluted with ethyl acetate (500 mL) and washed with sodium bicarbonate solution (200 mL), Organic layer was washed with water (2×200 mL), dried over anhydrous sodium sulphate and filtered. Volatiles were concentrated under reduced pressure to obtain the product as pale yellow liquid (17.0 g, 81.6%).

### Step-3: Preparation of 1,1′-ethane-1,2-diylbis(4-bromobenzene) (I-4)

To a suspension of iron powder (0.745 g, 0.01335 mol) in water (30 mL) was added copper chloride (0.120 g, 0.0012 mol). The reaction mixture was stirred for 20 minutes at room temperature then heated to 90° C. 4-bromo-4-chloromethyl benzene (5.0 g, 0.02433 mol) was added portion wise over 30 minutes. The reaction mixture was stirred at 90° C. for a period of 16 hours. The reaction was monitored by ¹H NMR until disappearance of starting materials was observed. The resulting reaction mass was cooled to room temperature, the precipitated solid was filtered and the crude product was purified through silica gel (230-400) column chromatography (in hexane) to obtain the product as a white solid (2.7 g, 35.36%).

### Step-4: Preparation of 4,4′-ethane-1,2-diyldibenzaldehyde (I-5)

To a solution of 1,1′-ethane-1,2-diylbis(4-bromobenzene) (0.35 g, 0.001 mol) in freshly distilled THF (7 mL) was added n-BuLi (1.36 mL, 1.6 M in THF, 0.0022 mol) dropwise at −78° C. over a period of 20 minutes with vigorous stirring. The molarity of the n-butyl lithium to be analyzed before starting the reaction. The stirring was continued at −78° C. for an additional 1 hour and dry DMF (0.8 mL) was added. The ice bath was removed after 5 minutes, slowly allowed to reach room temperature and the resulting reaction mass was stirred at room temperature for 2 hours. Reaction mass was quenched with water (20 mL) and extracted with ethyl acetate (3×50 mL). The combined organic extracts were washed with brine (50 mL) and dried over anhydrous sodium sulphate, filtered and concentrated under reduced pressure. The solid residue was purified by silica gel (230-400) column chromatography (15% ethyl acetate in hexane) to obtain the product as white solid (0.12 g, 48.97%).

### Step-5: Preparation of {4-[2-(4-hydroxymethyl-phenyl)-ethyl]-phenyl}-methanol (I-6)

To a solution of 4,4′-ethane-1,2-diyldibenzaldehyde (1.0 g, 0.0042 mol) in methanol (10 mL) was added sodium borohydride (0.388 g 0.0105 mol) at ice temperature. Resulting reaction mass was stirred at room temperature for 30 minutes. Reaction mass was quenched with saturated ammonium chloride solution (100 mL), aqueous layer was extracted with ethyl acetate (2×200 mL). Organic layer was washed with water (100 mL), dried over anhydrous sodium sulphate and filtered. Volatiles were concentrated under reduced pressure to obtain the product as off white solid (0.8 g, 80.0%).

### Step-6: Preparation of 1,1′-ethane-1,2-diylbis[4-(chloromethyl)benzene]-methane (I-7)

To a solution of {4-[2-(4-hydroxymethyl-phenyl)-ethyl]-phenyl}-methanol (1.5 g, 0.0061 mol) in dichloromethane (15 mL) was added thionyl chloride (2.3 mL, 0.0309 mol) slowly at ice temperature. Resulting reaction mass was stirred at room temperature for 10 minutes. Reaction mass was concentrated, diluted with ethyl acetate (200 mL) and washed with sodium bicarbonate solution (50 mL), Organic layer was washed with water (2×50 mL), dried over anhydrous sodium sulphate and filtered. Volatiles were concentrated under reduced pressure to obtain the product as off-white solid (1.2 g, 69.7%).

### Step-7: Preparation of 1,1′-ethane-1,2-diylbis[4-(iodomethyl)benzene] (I-8)

To a solution of 1,1′-ethane-1,2-diylbis[4-(chloromethyl)benzene] (0.2 g, 0.719 mmol) in acetone (10 mL) was added sodium iodide (0.43 g, 2.87 mmol) at room temperature. The resulting reaction mixture was stirred at reflux temperature over a period of 16 hours. The crude product obtained upon after the complete evaporation of the volatiles was diluted with ethyl acetate (100 mL). Organic layer was washed with water (2×50 mL), dried over anhydrous sodium sulphate and filtered. The volatiles were concentrated under reduced pressure. The solid residue obtained was purified by silica gel (230-400) column chromatography (2% Ethyl acetate in hexane) to obtain the product as white solid (0.15 g, 45.5%).

### Example 2

### Synthesis of racemic (±)-4,4′-((ethane-1,2-diylbis(4,1-phenylene))bis(methylene))bis(1-(hydroxymethyl)-5,7-dimethyl-2,3-dithia-5,7-diazabicyclo[2.2.2]octane-6,8-dione) (Compound 1)

Compound 1 was synthesized according to scheme II:

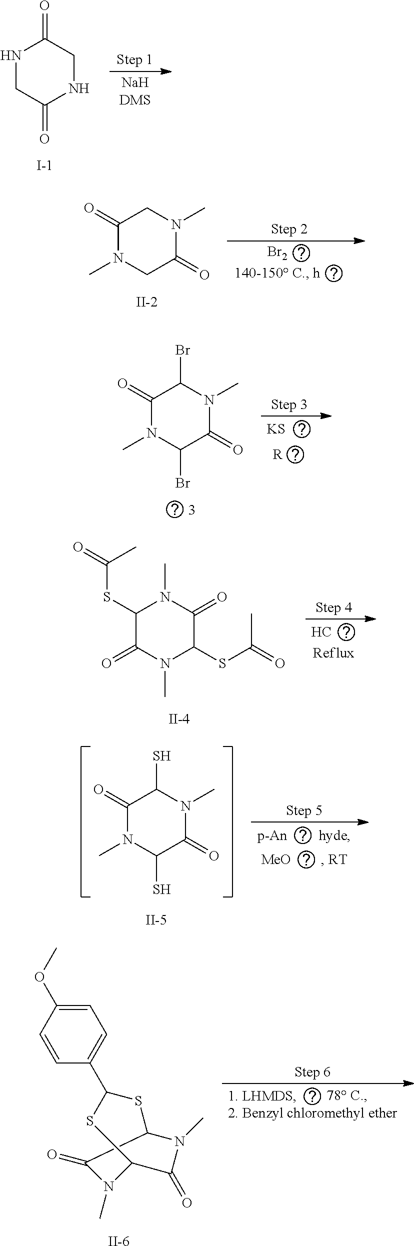

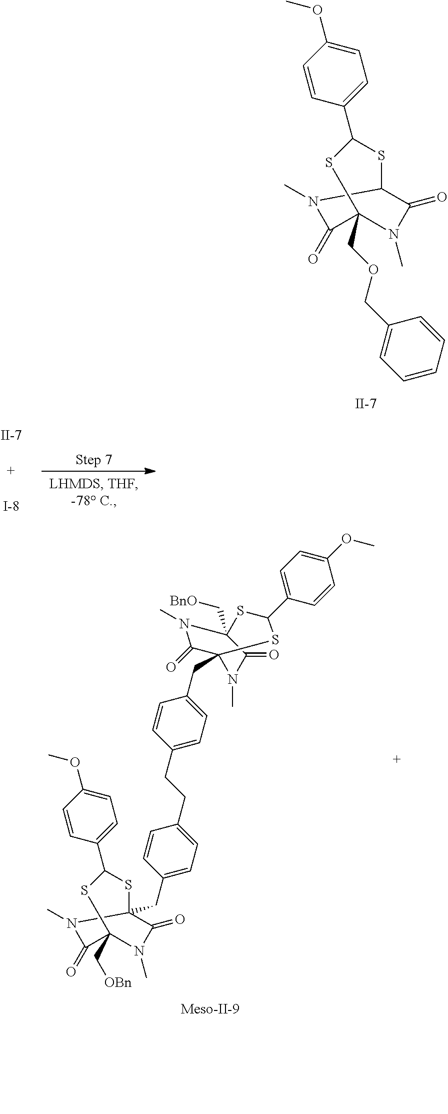

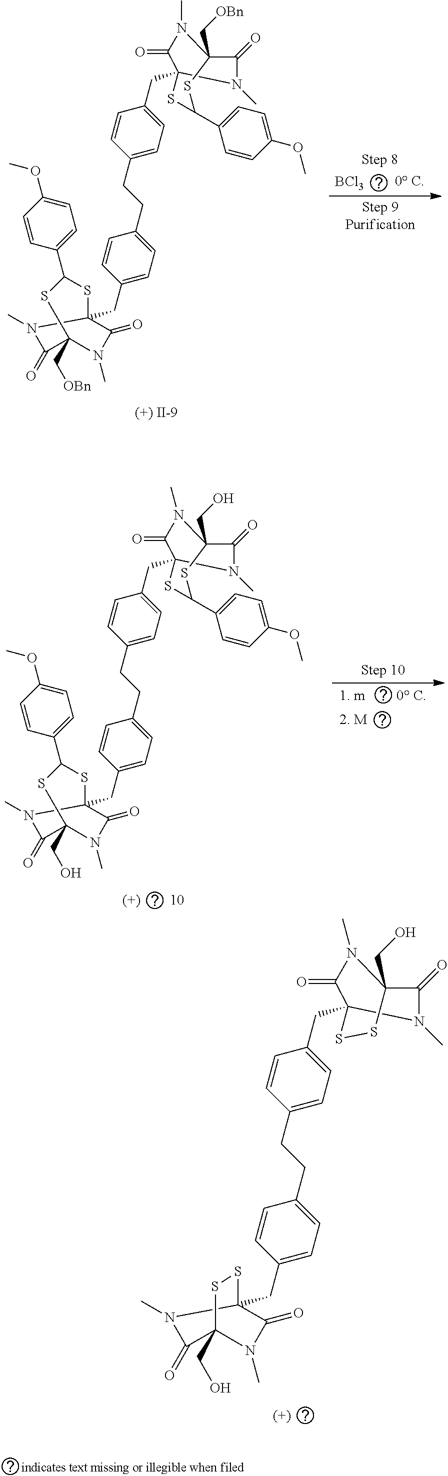

### Step 1: Preparation of 1,4-dimethylpperazine-2,5-dione (II-2)

To a stirred suspension of sodium hydride (1.311 Kg, 327.78 mol) in dry DMF (22 L) under argon atmosphere was added glycine anhydride (1.7 Kg, 148.99 mol) portion wise at ice temperature. Dimethyl sulfate (2.8 L, 297.98 mol) was slowly added drop wise with caution while stirring. The temperature was maintained at 0° C. throughout the addition, then allowed to reach room temperature and the mixture was stirred for 16 h. The resulting reaction mixture was quenched with methanol (4.2 L) and the volatiles were removed under reduced pressure to afford viscous slurry. The slurry was recrystallized by dichloromethane and diethyl ether to afford white solid that was isolated by filtration, washed with ice cold diethyl ether and then dried under high vacuum (0.6 kg, 28.0%).

### Step 2: Preparation of 1,4-dimethyl-2,5-diketopiperazine-3,6-dibromide (II-3)

To a solution of sarcosine anhydride II-2 (6.0 g, 0.0422 mol) in o-dichlorobenzene (72 mL) was heated to 150° C. and the reaction mixture was stirred vigorously. After complete dissolution of sarcosine anhydride, bromine (4.6 mL, 0.0929 mol) was added dropwise to the reaction mixture under continuous vigorous stirring and UV irradiation. The temperature of the oil bath was maintained at 140-150° C. Precipitation was observed while addition of bromine and precipitate was allowed to dissolve before more bromine was added. After completion of the bromine addition, the reaction was stirred at 140-150° C. for an additional 1 hour. The resulting hot dark-orange homogenous mixture was poured into a single neck round bottom flask and allowed to cool down to ambient temperature then 200 mL of hexane was added, the reaction mixture was cooled to −20° C. for 1 hour under nitrogen. The precipitate formed was isolated by decanting the hexane and dried under high vacuum overnight to obtain the product as fine glittery light-orange crystals (12.2 g, 96.0%).

### Step 3: Preparation of 1,4-dimethyl-2,5-diketopiperazine-3,6-bis-thioacetate (II-4)

To a solution of dibromide 11-3 (23.0 g, 0.0766 mol) in dichloromethane (230 mL) was added potassium thioacetate (35.8 g, 0.3143 mol) at room temperature. The reaction mixture was stirred for a period of 3 hours. Then reaction mixture was diluted with dichloromethane (500 mL) and washed with water (3×300 mL). The organic phase was dried over anhydrous sodium sulphate and filtered. The solvent was removed under reduced pressure to obtain the product as off-white solid (10 g, 45.0%).

### Step 4 & 5: Preparation of 3-(4-methoxyphenyl)-6,8-dimethyl-2,4-dithia-6,8-diazabicyclo[3.2.2]nonane-7,9-dione (II-6)

To a solution of dithioacetate 11-4 (6.0 g, 0.0206 mol) in methanol (60 mL) was added HCl in methanol (1.25M, 170 mL, 0.0206 mol). The reaction mixture was then refluxed under nitrogen atmosphere for a period of 5 hours. After that, the reaction was cooled to room temperature with continuous stirring. P-anisaldehyde (7.5 mL, 0.0620 mol) was added and the reaction mixture was stirred for another 18 h at room temperature. The white precipitate formed was isolated by filtration and dried overnight at room temperature under high vacuum (3.48 g, 52.1%).

### Step 6: Preparation of 1-(benzyloxymethyl)-3-(4-methoxyphenyl)-6,8-dimethyl-2,4-dithia-6,8-diazabicyclo[3.2.2]nonane-7,9-dione (II-7)

To a solution of 3-(4-methoxy-phenyl)-6,8-dimethyl-2,4-dithia-6,8-diaza-bicyclo[3.2.2]nonane-7,9-dione II-6 (3.0 g, 0.0092 mol) in dry THF (240 mL) at −78° C., LHMDS (1.0 M solution in THF) (11.0 mL, 0.0110 mol) was added dropwise over a period of 10 minutes with stirring. Benzyl chloromethyl ether (5 mL, 0.0369 mol) was added to the reaction mass and the resulting reaction mixture was allowed to stir −78° C. over a period of 1 h. The reaction mixture was quenched with saturated ammonium chloride solution (200 mL) and the mixture was extracted with ethyl acetate (2×300 mL). The combined organic extracts were washed with water (2×100 mL) dried over anhydrous sodium sulphate, filtered and concentrated under reduced pressure. The concentrated product was dissolved in dichloromethane (4V), precipitated with diethyl ether (20 V) and filtered to obtain the product as white solid (1.3 g, 32.0%).

### Step 7: Preparation of 5,5′-((ethane-1,2-diylbis(4,1-phenylene))bis(methylene))bis(1-((benzyloxy)methyl)-3-(4-methoxyphenyl)-6,8-dimethyl-2,4-dithia-6,8-diazabicyclo[3.2.2]nonane-7,9-dione) (II-9)

A solution of (S)-1-benzyloxymethyl-3-(4-methoxy-phenyl)-6,8-dimethyl-2,4-dithia-6,8-diaza-bicyclo[3.2.2]nonane-7,9-dione 11-7 (5.92 g, 0.0086 mol) in dry THF (360 mL) was cooled to −78° C. LHMDS (1.0 M solution in THF) (21.6 mL, 0.0216 mol) was added dropwise over a period of 15 minutes with stirring. 1,1′-ethane-1,2-diylbis[4-(iodomethyl)benzene] (2.0 g, 0.0043 mol) in THF (20 mL) was added to the reaction mass and the resulting reaction mixture was stirred at the same temperature over a period of 1 hour. After consumption of starting material was observed, the reaction mixture was quenched with saturated ammonium chloride solution (200 mL) and the mixture was extracted with ethyl acetate (2×300 mL). The combined organic extracts were washed with water (2×100 mL) dried over anhydrous sodium sulphate, filtered and concentrated under reduced pressure. The crude product obtained was washed with diethyl ether to obtain the product as off-white solid (2.6 g, 55.3%).

### Step 8: Preparation of 5,5′-(Ethane-1,2-diylbis(4,1-phenylene))bis(methylene)) bis(1-(hydroxymethyl)-3-(4-methoxyphenyl)-6,8-dimethyl-2,4-dithia-6,8-diazabicyclo[3.2.2] nonane-7,9-dione) (II-10)

To a solution of compound II-9 (2.6 g, 0.23 mmol) in dichloromethane (52 mL) was added boron trichloride (6.6 mL, 1.0 M in DCM, 0.59 mmol) at ice temperature. Resulting reaction mass was stirred at ice temperature for 15 minutes. Reaction mass was added to ice cold water (100 mL) and extracted with dichloromethane (250 mL). The aqueous layer was re-extracted with dichloromethane (2×100 mL). The combined organic extracts were dried over anhydrous sodium sulphate, filtered and concentrated under reduced pressure. The crude compound was purified by column chromatography to remove non polar impurities. Then isomers were separated on silica gel (230-400) column using gradient of ethyl acetate (2.5, 5.0, 7.5 and 10%) in dichloromethane. The separation of isomers was achieved after 2-3 column purifications yielding pure meso-II-10 and (±)-II-10 (750 mg of each isomer).

### Step 9: Preparation of (±)-4,4′-((ethane-1,2-diylbis(4,1-phenylene))bis(methylene))bis(1-(hydroxymethyl)-5,7-dimethyl-2,3-dithia-5,7-diazabicyclo[2.2.2]octane-6,8-dione) (Compound 1)

To a solution of compound (±)-II-10 (3.3 g, 0.36 mmol) in dichloromethane (5.77 L) was added m-chloro peroxy benzoic acid (3.05 g, 1.1 mmol, 65%) at ice temperature. After 30 minutes ice bath was removed, dimethyl sulfide (1.07 mL, 1.47 mmol) and trifluoro acetic acid (21.57 mL) was added at room temperature. The resulting reaction mixture was stirred at room temperature for 16 hours. The reaction mass was quenched with aqueous saturated sodium bicarbonate solution (2.47 L) and the organic layer was separated. The aqueous layer was again extracted with dichloromethane (2.5 L), the combined organic layers were dried over anhydrous sodium sulphate and concentrated. The crude product obtained upon complete evaporation of volatiles was purified by silica gel (230-400) column chromatography (1.2% Methanol in dichloromethane) to obtain the product as off-white solid (1.3 g, 53.5%, 86% HPLC purity). The product was further purified by preparative HPLC to obtain the racemic product (compound 1) as white solid (650 mg, 50.0%).

### Example 3

### Tumor Xenograph Studies

Mouse xenograft study was conducted to evaluate the primary endpoint of efficacy (reduction in tumor volume of >50% as compared to control) and the secondary endpoint of survival (Kaplan-Meier). Tumors were established in the flank region and grown to about 100 mm³ The mice were treated with compound 1 in the 786-O xenografts (renal cell carcinoma) at 1 mg/kg given IV. The compound was injected every fourth day for 45 days (a total of 11 injections, see following table).

| Injection | | | Mode |
|---|---|---|---|
| Day 0 | 1 | mg/kg | i.v. |
| Day 6 | 1 | mg/kg | i.v. |
| Day 11 | 0.5 | mg/kg | i.v. |
| Day 21 | 0.5 | mg/kg | i.p. |
| Day 26 | 0.5 | mg/kg | i.p. |
| Day 31 | 0.5 | mg/kg | i.p. |
| Day 36 | 0.5 | mg/kg | i.p. |
| Day 46 | 0.5 | mg/kg | i.p. |

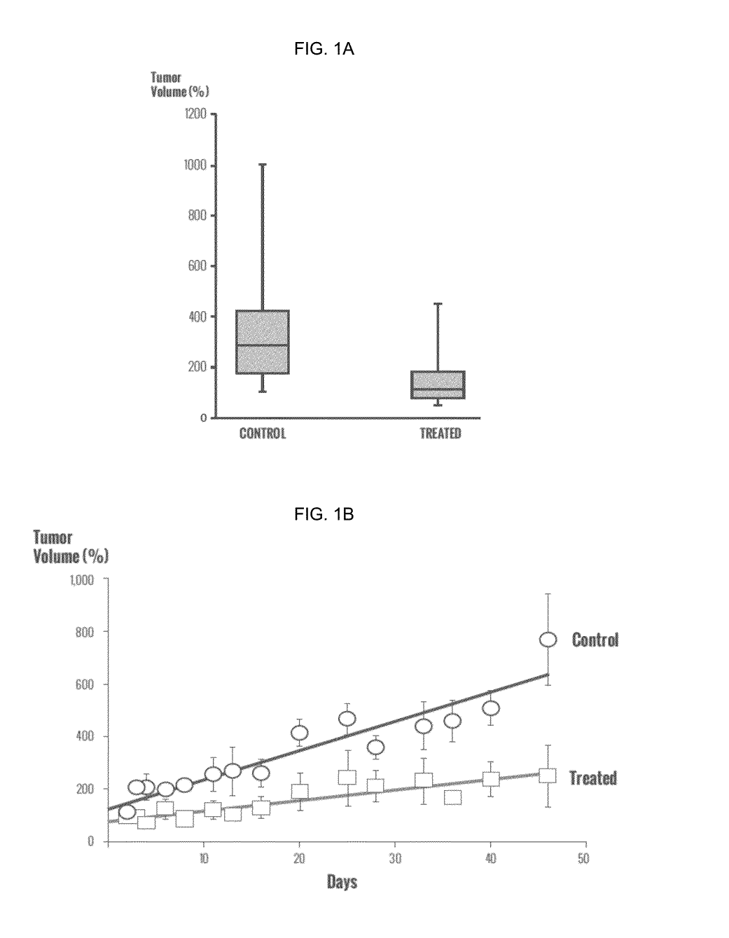

All tumors were imaged at the end of the study and harvested for histopathology studies. Percentages of tumor volumes in control and compound 1—treated mice measured throughout the entire duration of the experiments. These data are shown in FIGS. 1A and 1B. Error bars are ±SEM of each experimental group. FIG. 1A demonstrates tumors in mice treated with compound 1 were smaller (median volume increase: 113%) than those of control mice group (288%, *P<0.01, t-test). FIG. 1B shows a box-whisker diagram of the percentages of tumor volumes measured throughout the 46 days of the experiment with boxes representing the upper and lower quartiles and median and error bars showing maximum and minimum tumor volumes.

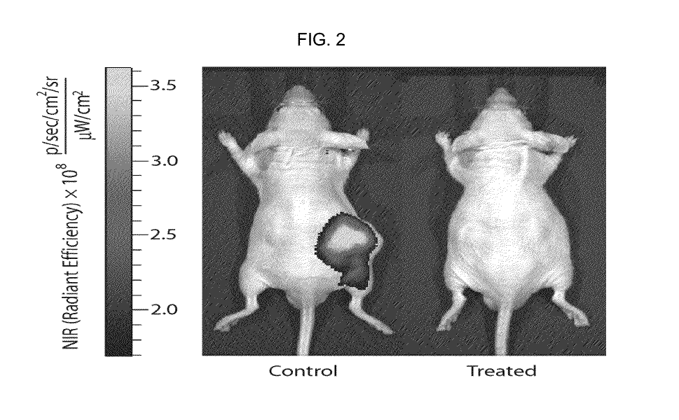

After the last treatment with compound 1, mice were injected with the tumor-targeting near-infrared (NIR) contrast agent IR-783, anesthetized and imaged on a Xenogen IVIS 200 small animal imager. The images taken from the dorsal (spine) side are shown in FIG. 2. The fluorescence output was processed with Living Image software with one representative sample for each group presented above. Mice from the group treated with compound 1 show significantly lower intensity of the NIR signal originating from the tumor-accumulated contrast agent as compared to the control group that was not treated with compound 1 (FIG. 2). The weight loss did not exceed 5-8% during the 45 day experiment.

### Example 4

### Maximum Tolerated Dose Studies

Three groups of three animals (male BALB/c mice) were administered increasing doses of compound 1 every other day for 14 days and the animals checked daily for the signs of moribundity, mortality, vocalization, and gross weight or body shape abnormalities. Dose escalation of compound 1 was performed according to the “modified Fibonacci search” scheme, compound 1 (x=2 mg/kg) was dosed at 4, 6.6, 10, 14, 24 and 32 mg/kg on Days 0, 2, 4, 6, 8 and 10. The maximum tolderated dose (MTD) was determined and no signs of toxicity were observed at 14 mg/kg. At 24 mg/kg dose, one animal exhibited signs of toxicity. An average MTD of 21.1 mg/kg for the three groups combined was determined from this study.

| Dose (mg/kg) | Group 1 | Group 2 | Group 3 |
|---|---|---|---|
| 4 | 0 | 0 | 0 |
| 6.6 | 0 | 0 | 0 |
| 10 | 0 | 0 | 0 |
| 14 | 0 | 0 | 0 |
| 24 | 0 | 1 | 1 |
| 32 | 3 | 3 | 3 |
| MTD | 25.3 | 19 | 19 |
| Average MTD | | 21.1* | |

### Example 5

### Rodent ADME and Pharmacokinetics Studies

The Log D_{7.4} value of compound 1 was measured to be 2.69 under standard experimental conditions. Inhibition of multiple CYP was measured for compound 1 using standard CYP inhibition assays. Each assay showed an IC₅₀>5 μM concentration. Plasma protein binding data for compound 1 was measured and the compound was determined to be highly protein bound indicating. Human microsome stability of compound 1 in the presence of co-factor showed a short T_{1/2}, and human microsome stability studies of compound 1 indicated favorable in vitro clearance.

IV Dosing:

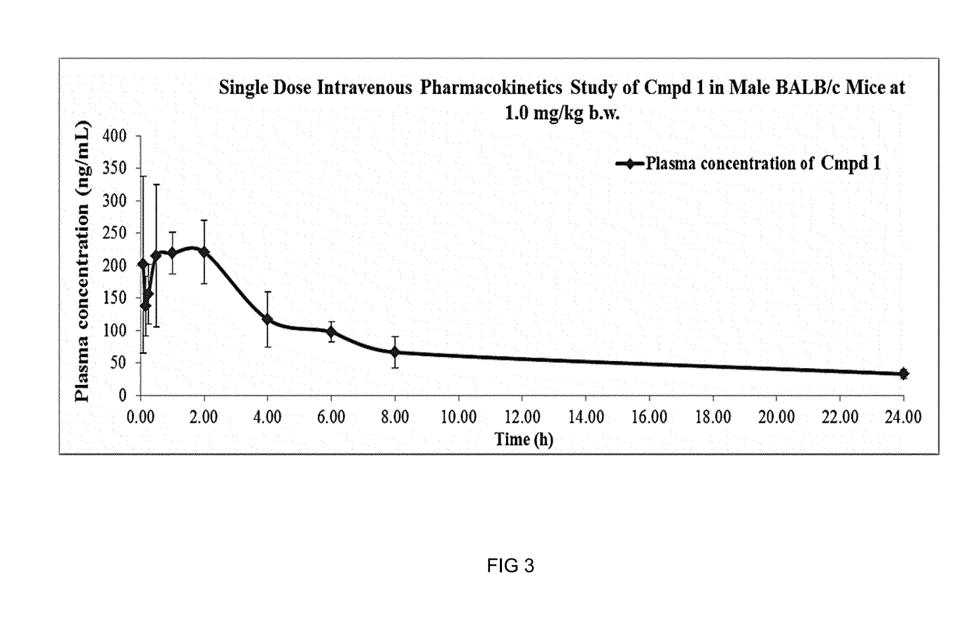

Non-fasted animals (Male and Female BALB/c mice or SD rats, ten groups, three animals per group) were administered with compound 1 in vehicle (5% (v/v) N,N-Dimethylacetamide, 50% (v/v) of 60% (w/v) Hydroxypropyl beta-Cyclodextrin, 45% (v/v) Sterile water for injection) by intravenous route with various doses 0.5, 1, 2.5, and 5 mg/kg body weight at dose volume of 10 mL/kg body weight. Under mild isoflurane anesthesia, blood specimens were collected into pre-labeled tubes containing anticoagulant (K2EDTA—2 mg/mL blood) at various time points (0.08, 0.16, 0.25, 0.50, 1.00, 2.00, 4.00, 6.00, 8.00 and 24.00 hours). Collected blood specimens were centrifuged at 4000 rpm, 4° C. for 10 minutes and plasma was separated and stored at −80° C. until analysis. The data from these experiments are displayed in FIG. 3-8.

FIG. 3 shows the plasma concentration of compound 1 of the disclosure over time in an intravenous pharmacokinetic study of male BALB/c mice (1.0 mg/kg b.w.).

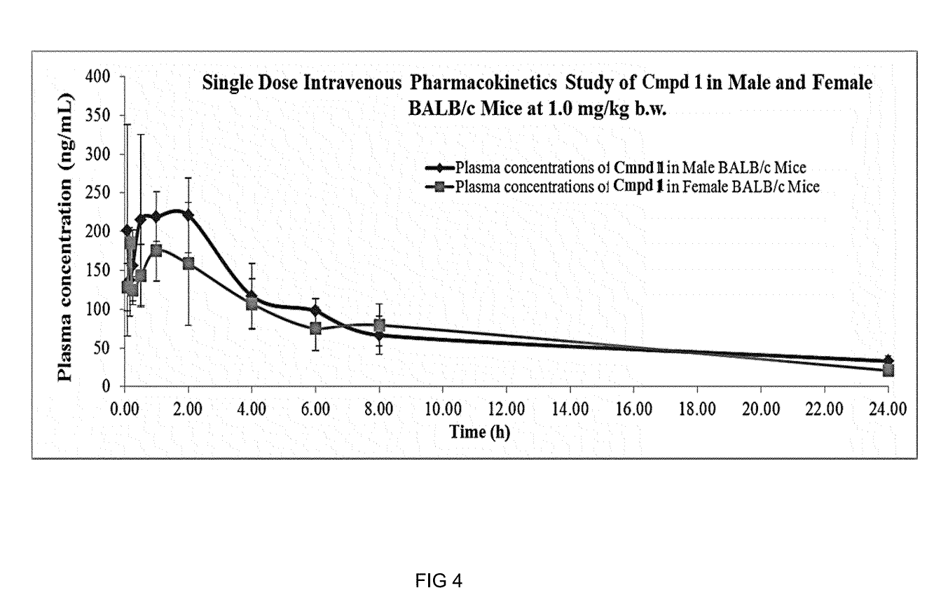

FIG. 4 shows the plasma concentration of compound 1 of the disclosure over time in an intravenous pharmacokinetic study of male and female BALB/c mice (1.0 mg/kg b.w.). Animals were observed for any adverse symptoms after dosing of compound 1. All the animals were found to be normal up to the observed period of 24 hours post dose.

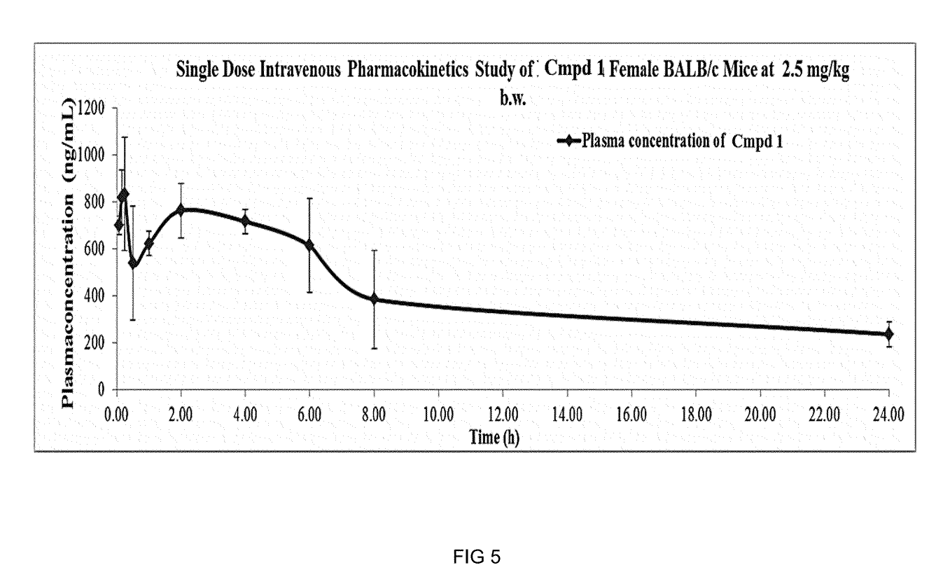

FIG. 5 shows the plasma concentration of compound 1 of the disclosure over time in an intravenous pharmacokinetic study of female BALB/c mice (2.5 mg/kg b.w).

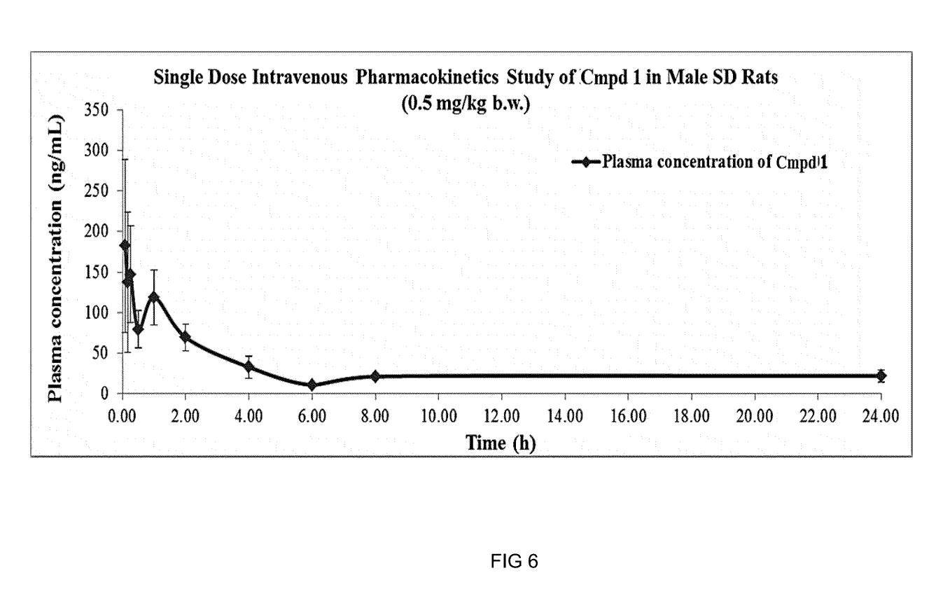

FIG. 6 shows the plasma concentration of compound 1 of the disclosure over time in an intravenous pharmacokinetic study of male SD rats (0.5 mg/kg b.w.).

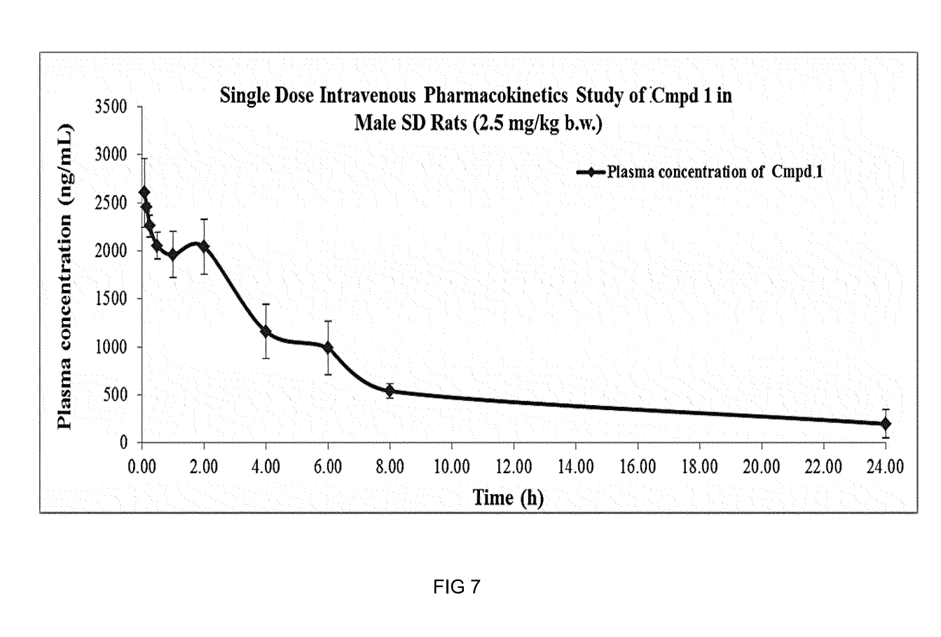

FIG. 7 shows the plasma concentration of compound 1 of the disclosure over time in an intravenous pharmacokinetic study of male SD rats (2.5 mg/kg b.w.).

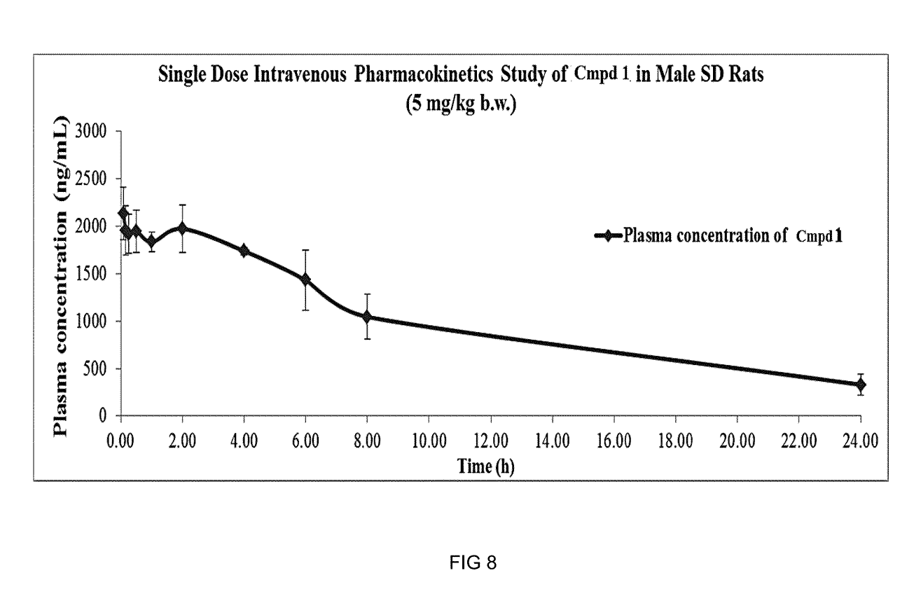

FIG. 8 shows the plasma concentration of compound 1 of the disclosure over time in an intravenous pharmacokinetic study of male SD rats (5 mg/kg b.w.).

Oral Dosing:

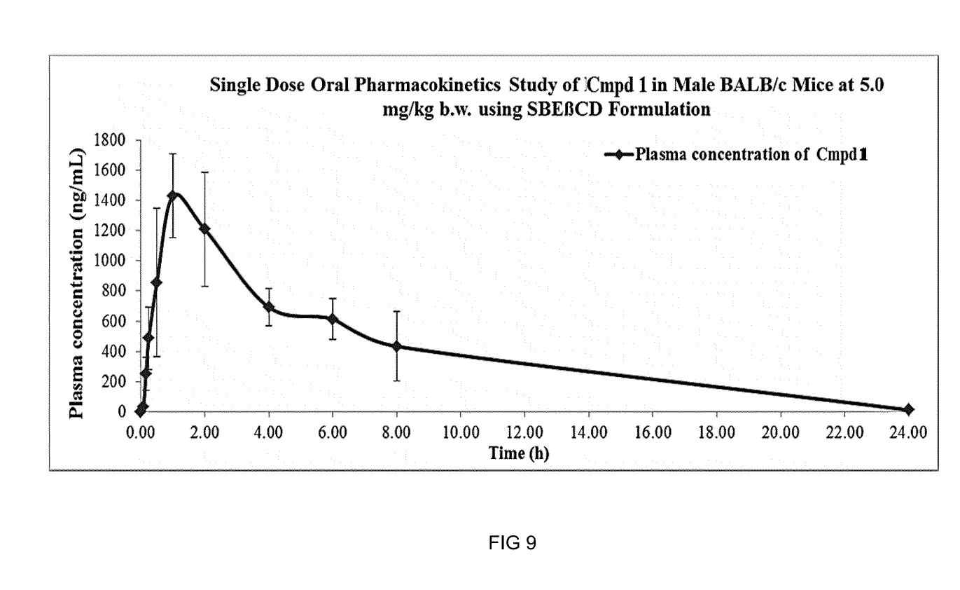

Fasted animals (Male and Female BALB/c mice or SD rats, ten groups, three animals per group) were administered with compound 1 in vehicle (5% (v/v) N,N-Dimethylacetamide, 50% (v/v) of 60% (w/v) Sulfobutyl ether beta-cyclodextrin (SBEβCD) and 45% (v/v) Sterile water for injection) by oral route with a dose of 2.5, 5, and 10 mg/kg body weight at dose volume of 10 mL/kg b.w. Under mild isoflurane anesthesia, blood specimen were collected into pre-labeled tubes containing anticoagulant (K2EDTA—2 mg/mL blood) at various time points (0.08, 0.16, 0.25, 0.50, 1.00, 2.00, 4.00, 6.00, 8.00 and 24.00 hours). Collected blood specimens were centrifuged at 4000 rpm, 4° C. for 10 minutes and plasma was separated and stored at −80° C. until analysis. The data from these experiments are displayed in FIG. 9-13.

FIG. 9 shows the plasma concentration of compound 1 of the disclosure over time in an oral pharmacokinetic study of male BALB/c mice (5 mg/kg b.w.) with SBEβCD formulation.

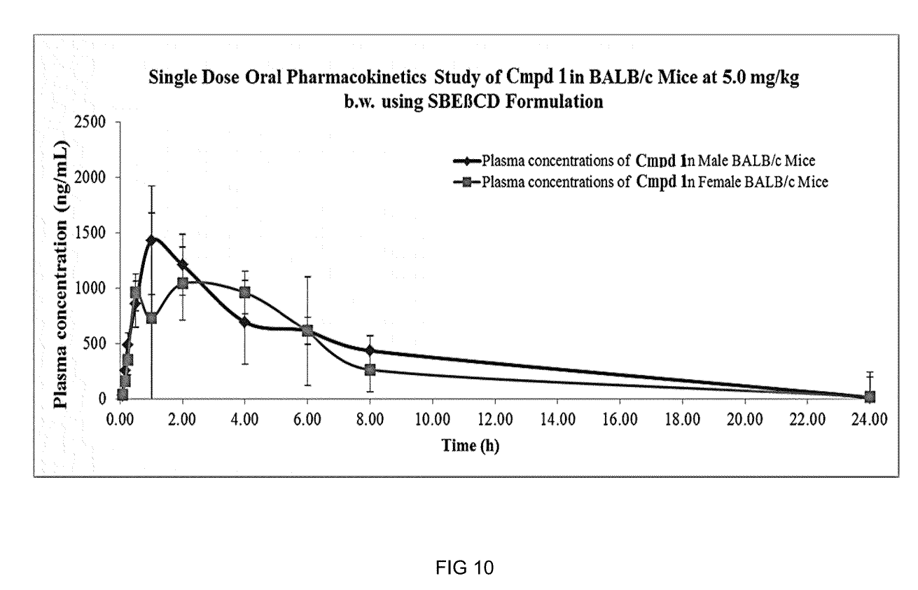

FIG. 10 shows the plasma concentration of compound 1 of the disclosure over time in an oral pharmacokinetic study of male and female BALB/c mice (5 mg/kg b.w.) with SBEβCD formulation.

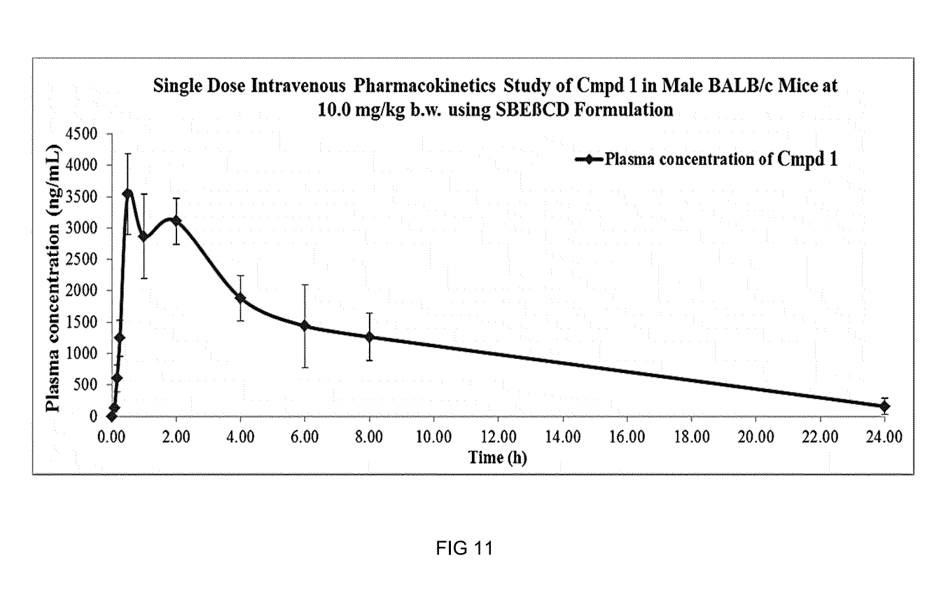

FIG. 11 shows the plasma concentration of compound 1 of the disclosure over time in an oral pharmacokinetic study of male BALB/c mice (10 mg/kg b.w.) with SBEβCD formulation.

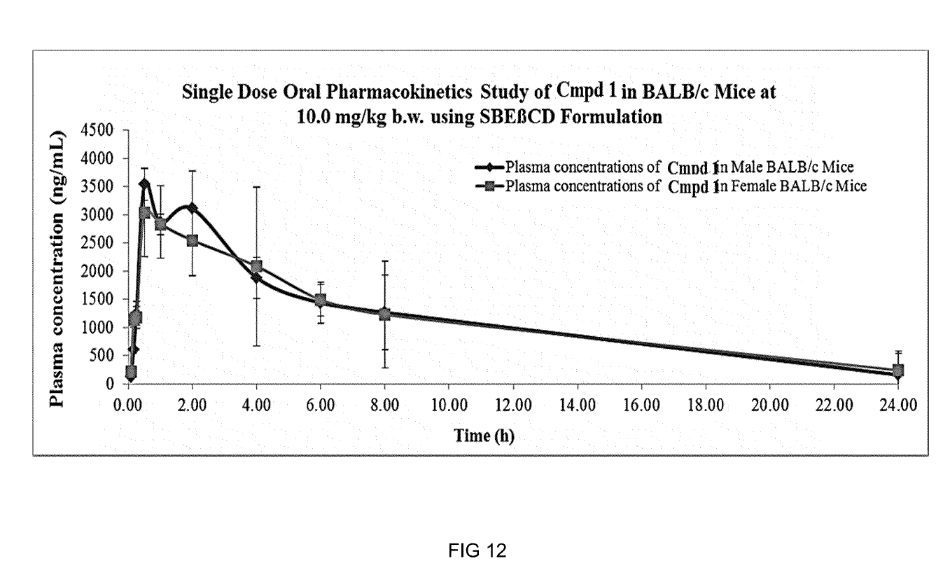

FIG. 12 shows the plasma concentration of compound 1 of the disclosure over time in an oral pharmacokinetic study of male and female BALB/c mice (10 mg/kg b.w.) with SBEβCD formulation.

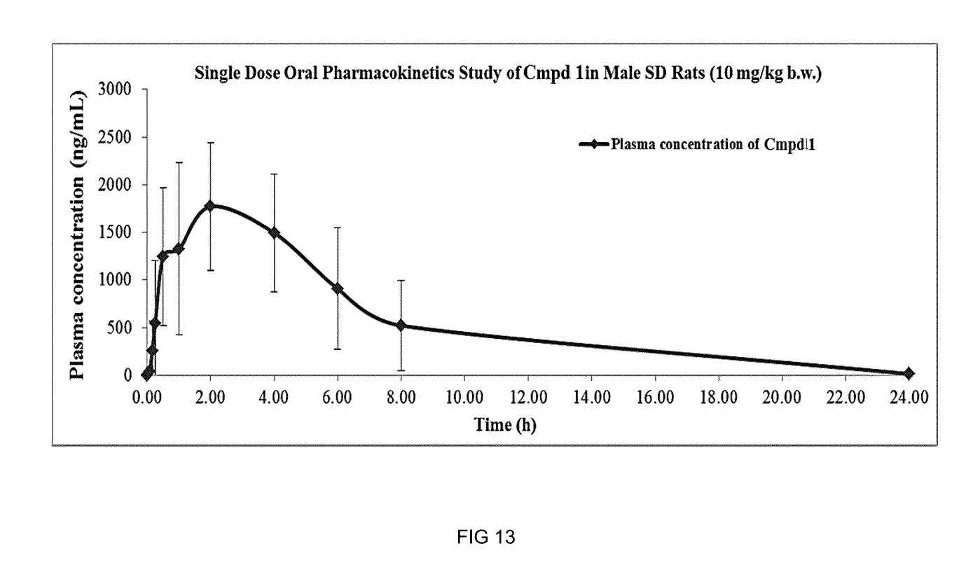

FIG. 13 shows the plasma concentration of compound 1 of the disclosure over time in an oral pharmacokinetic study of male SD rats (10 mg/kg b.w.) with SBEβCD formulation. Additionally, bioavailability of 28% was calculated at 5 mg/kg b.w. in Male SD rats.

### Example 6

### Evaluation of Binding Affinity and Specificity of Designed ETP Analogs

The binding affinities of the synthetic ETP analogues disclosed herein is toward its target, the p300/CBP CH1 domain that has been expressed as a GST fusion protein is determined. A Fluorescence polarization assay with dye-labeled HIF-1α C-TAD is used to determine the thermodynamic binding affinities of the designed ETP toward the p300/CBP CH1 domain. Chetomin is used as a control and the binding affinities of the synthesized analogues and chetimin are compared. In addition, skeletal analogs of ETP without a sulfide bridge, are used as negative controls.

### Example 7

### Fluorescence Polarization (FP) Competition Experiments

HIF-1α peptide (sequence H2N-SMDESGLPQLTSYDCEVNAPIQGS-RNLLQGEELLRALDQVN-CONH2), synthesized on the solid phase using fluorenylmethoxycarbonyl (Fmoc) strategy and labeled with fluorescein, is used as the probe in competition fluorescence polarization (FP) experiments. Fluorescence polarization experiments will be performed using a multi-label plate reader equipped with polarized filters and optical modules for fluorescein (excitation, 488 nm; emission, 515 nm). First, the binding dissociation constant (Kd) of fluorescein-C-TAD for P300 CH1 is determined by a direct binding FP assay. Competition FP assays are conducted in 96-well plates with final assay concentrations of p300 CH1 and fluorescein-C-TAD probe in the range of 1 μM concentration in assay buffer that contains 20 mM phosphate, 1 mM EDTA, 50 mM NaCl, at pH 7.4.

Final assay concentrations of ETP analogs, added as a 50-100 mM stock solution in DMSO (quantified by weight of the compound), range from 40 mM to 0.0002 uM; the final assay concentration of DMSO is 0.4% (v/v). Assay plates are typically incubated in the dark for ˜3 h at room temperature before being analyzed by the plate reader. Raw competition FP data are converted to percent inhibition of the fluorescein-C-TAD/p300 CH1 interaction, and binding data are fit by using the single-site competition binding model to determine IC₅₀ values and associated confidence intervals. Inhibitor dissociation constant (Ki) values are calculated from IC₅₀ values as described in the literature.

### Example 8

### Analysis of Transcription Inhibition: (a) HIF-1-CBP/p300 Complex

To test the ability of the newly synthesized analogues to inhibit the activity of HIF-1-CBP/p300 complex in cell-based assays in vitro, three assays are utilized:

Analysis of Hypoxia-Inducible Promoter Activity with Luciferase Assays.

A luciferase based assay is used to screen the designed analogues to find the most potent inhibitors of the hypoxia-inducible transcription. To measure gene expression under hypoxia conditions, the two groups of stably transfected cells are typically incubated for 6 to 18 hours, both under well oxygenated and hypoxia conditions. After 2 hours of reoxygenation, cell lysates are prepared and the luciferase activity is measured with a luminometer, as previously described. The ratio of the luciferase activity under hypoxic vs. normoxic conditions provides fold induction in hypoxia, used to measure the activity of hypoxia-inducible promoter.

Real-Time Quantitative RT-PCR.

Cell culture, isolation of mRNA and real-time quantitative RT-PCR analysis are performed using previously published protocols.

### Example 9

### Evaluation of the Genome-Wide Effects of Synthetic ETP Analogues

A fundamental proposed advantage of transcriptional inhibitors, such as ETPs, is that they can allosterically target the transcription factor complex, which may translate into fewer off-target effects than direct-acting inhibitors. The specificity of the designed analogues is tested on cellular transcriptome with gene expression profiling (GEP). GEP is also valuable because it can allow assessment of the impact of the compound on multiple signaling pathways and downstream targets of hypoxia-inducible transcription. The effect on transcriptional activity of RAR, SREBP2, SRC-1 and other genes is evaluated, to test potential antagonists for downregulation of global gene expression. In the typical experiment, cells of choice are plated and incubated for 20-26 h with compounds and controls in two concentrations. Hypoxia conditions are induced for 12 h and total RNA is collected. After quality testing, the total RNA is subjected to Affymetrix protocols. Affymetrix ST1 Human Gene Arrays are employed in the analysis.

What is claimed is:

1. A compound of Formula 1: 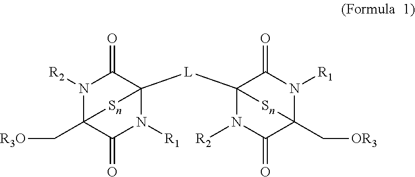
or a pharmaceutically acceptable salt thereof, wherein
each n is independently 1 or 2;
each R₁ and R₂ is independently selected from the group consisting of H, optionally substituted C₁-C₆ alkyl, optionally substituted C₃-C₇ heterocycloalkyl and optionally substituted C₃-C₇ cycloalkyl;
each R₃ is independently selected from the group consisting of H, optionally substituted C₁-C₆ alkyl, PEG, —C(O)R₄, —C(O)OR₄, —C(O)NR₄, and —S(O)₂R₄;
each R₄ when present is independently selected from the group consisting of optionally substituted C₁-C₆ alkyl, optionally substituted C₃-C₇ heterocycloalkyl, optionally substituted C₃-C₇ cycloalkyl, optionally substituted aryl, and optionally substituted heteroaryl;
L is: 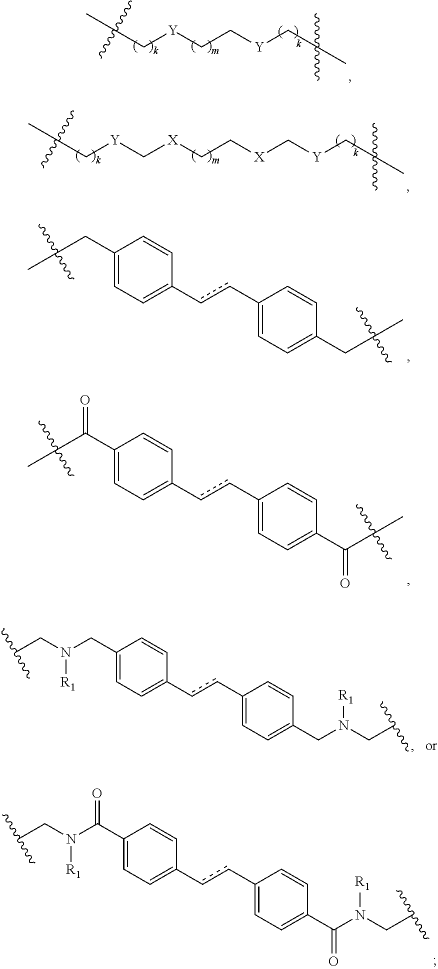
each X is independently NR₅ or O;
each Y is independently selected from the group consisting of a bond, methylene, aryl and heteroaryl; [symbol]
is a single or double bond;
each R₅ is independently selected from the group consisting of H, optionally
substituted C₁-C₆ alkyl, optionally substituted C₃-C₇ heterocycloalkyl and optionally substituted C₃-C₇ cycloalkyl;
each k is independently 0, 1, 2, or 3;
and m is 1, 2, 3, or 4.

2. The compound of claim 1, wherein each R₁ and each R₂ is independently selected from the group consisting of H, optionally substituted C₁-C₆ alkyl, and optionally substituted C₃-C₇ cycloalkyl.

3. (canceled)

4. The compound of claim 2, wherein each R₃ is independently selected from the group consisting of H, —C(O)R₄, —C(O)OR₄, —C(O)NR₄.

5. (canceled)

6. The compound of claim 4, wherein L is 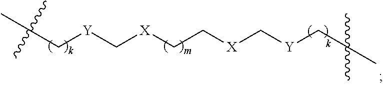
each X is independently NR₅ or O;
each R₅ is independently selected from the group consisting of H, optionally substituted C₁-C₆ alkyl, optionally substituted C₃-C₇ heterocycloalkyl and optionally substituted C₃-C₇ cycloalkyl;
each Y is independently selected from the group consisting of a bond, methylene, aryl and heteroaryl;
each k is independently 0, 1, 2, or 3;
and m is 1, 2, 3, or 4.

7. The compound of claim 6, wherein L is 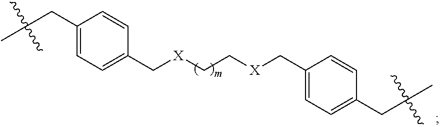
each X is independently NR₅ or O; and m is 1, 2, 3, or 4.

8. The compound of claim 6, wherein L is 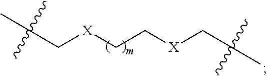
and m is 1, 2, 3, or 4.

9. (canceled)

10. (canceled)

11. (canceled)

12. The compound of claim 4, wherein L is

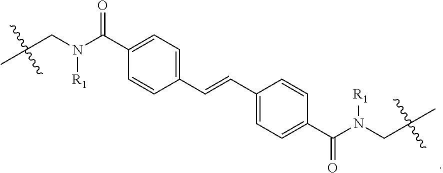

13. The compound of claim 4, wherein L is

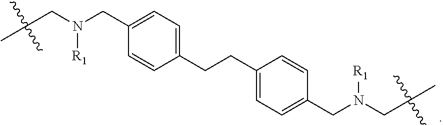

14. (canceled)

15. The compound of claim 4, wherein L is

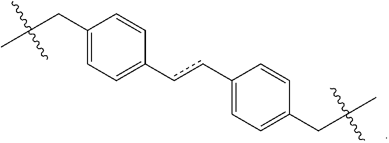

16. The compound of claim 4, wherein L is

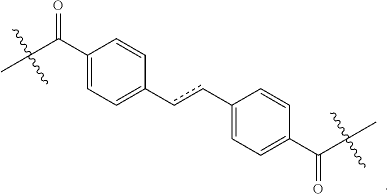

17. The compound of claim 4, wherein L is 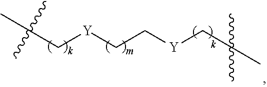
each Y is independently selected from the group consisting of a bond, methylene, aryl and heteroaryl;
each k is independently 0, 1, 2, or 3;
and m is 1, 2, 3, or 4.

18. The compound of claim 17, wherein each Y is independently aryl or heteroaryl.

19. (canceled)

20. (canceled)

21. The compound of claim 18, wherein each k is 1, and m is 1.

22. (canceled)

23. The compound of claim 1, having the structure of Formula 1a:

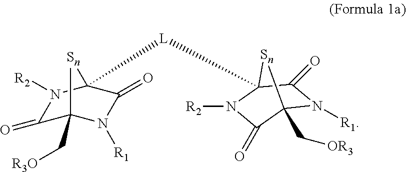

24. (canceled)

25. (canceled)

26. A compound selected from the group consisting of:

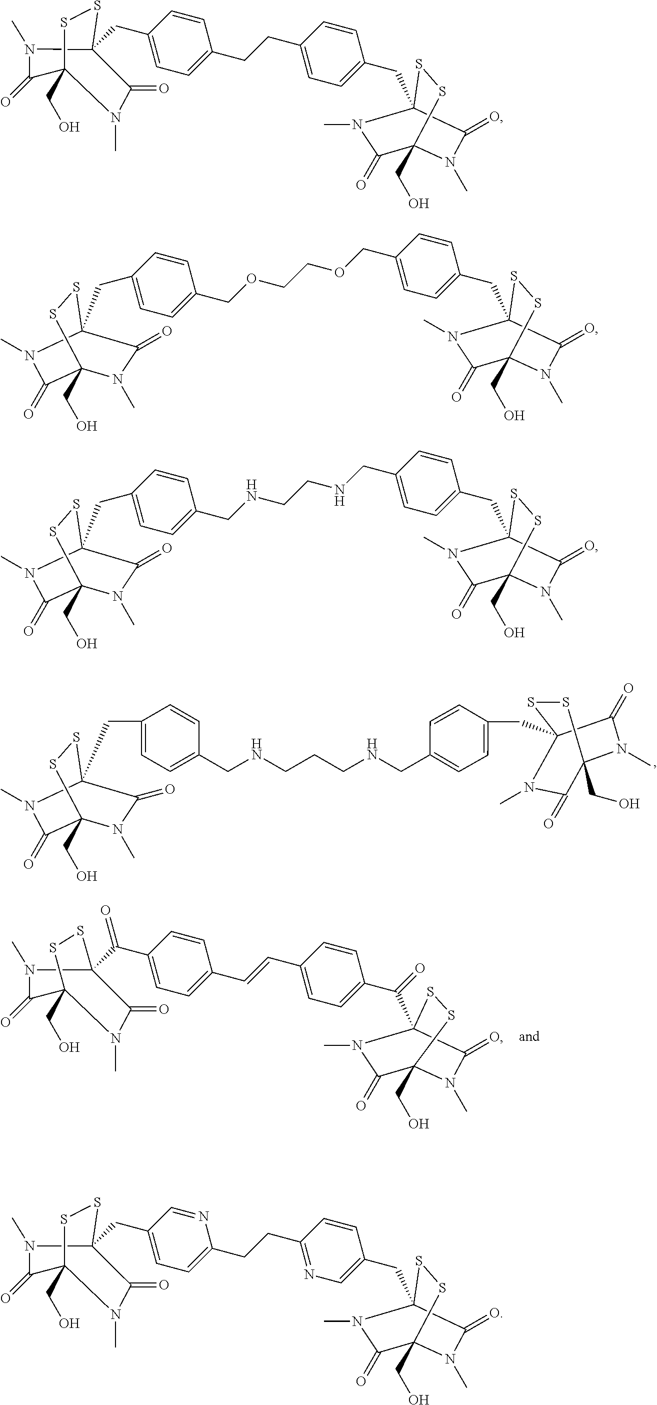

27. A pharmaceutical composition comprising at least one compound according to claim 1.

28. (canceled)

29. A method for treating a solid cancer, a blood cancer, or a breast cancer comprising: administering to a subject in need thereof an effective amount of at least one compound according to claim 1.

30. (canceled)

31. (canceled)

32. (canceled)

33. (canceled)

34. A method for treating a viral infection, wherein the viral infection is an infection of human papilloma virus (HPV), hepatitis C (HCV), Hep B, or adenovirus, comprising: administering to a subject in need thereof an effective amount of at least one compound according to claim 1.

35. (canceled)

36. The method of claim 29, further comprising administering an additional anti-cancer and/or cytotoxic agent.

37. The method of claim 34, further comprising administering an additional antiviral agent.
